(19) United States
(12) Patent Application Publication
(10) Pub. No.: US 2010/0291137 A1
(43) Pub. Date: Nov. 18, 2010

(54) METHOD FOR REFOLDING NEISSERIAL NSPA PROTEIN

(75) Inventors: Ralph Biemans, Rixensart (BE); Martine Bos, Utrecht (NL); Philippe Denoel, Rixensart (BE); Christiane Feron, Rixensart (BE); Carine Goraj, Rixensart (BE); Jan Poolman, Rixensart (BE); Vincent Weynants, Rixensart (BE); Johannes Petrus Maria Tommassen, Utrecht (NL)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: GLAXOSMITHKLINE BIOLOGICALS SA, Rixensart (BE)

(21) Appl. No.: 12/693,271

(22) Filed: Jan. 25, 2010

Related U.S. Application Data
(63) Continuation of application No. 10/525,889, filed on Sep. 10, 2007, now abandoned.

(30) Foreign Application Priority Data
Aug. 30, 2002 (GB) 0220197.8

(51) Int. Cl. A61K 39/095; C07K 14/22; A61K 39/116; A61K 39/385; A61P 31/04

(52) U.S. Cl. 424/197.11; 530/350; 424/249.1; 424/250.1; 424/203.1

(57) ABSTRACT

The present invention provides an isolated refolded NspA protein, and a method of preparing it.

### CROSS-REFERENCE TO RELATED APPLICATIONS

This application is a Continuation of U.S. application Ser. No. 10/525,889 filed Sep. 10, 2007; which was filed pursuant to 35 U.S.C. §371 as a U.S, National Phase Application of International Patent Application No. PCT/EP03/10085 filed Aug. 28, 2003; which claims priority from Great Britain Application No. 0220197.8 filed in the United Kingdom on Aug. 30, 2002.

### FIELD OF THE INVENTION

The present invention relates to a method of refolding NspA protein—an outer membrane protein of Neisseria meningitidis organisms, to such refolded proteins, pharmaceutical compositions comprising them, and their use in the treatment, prevention and diagnosis of bacterial infections, such as Neisserial infections, and particularly, but not exclusively, Neisseria meningitidis and/or Neisseria gonorrhoeae.

### BACKGROUND OF THE INVENTION

Neisserial strains of bacteria are the causative agents for a number of human pathologies, against which there is a need for effective vaccines to be developed. In particular Neisseria gonorrhoeae and Neisseria meningitidis cause pathologies which could be treated by vaccination.

Neisseria gonorrhoeae is the etiologic agent of gonorrhea, one of the most frequently reported sexually transmitted diseases in the world with an estimated annual incidence of 62 million cases (Gerbase et al 1998 Lancet 351; (Suppl 3)2-4). The clinical manifestations of gonorrhea include inflammation of the mucus membranes of the urogenital tract, throat or rectum and neonatal eye infections. Ascending gonococcal infections in women can lead to infertility, ectopic pregnancy, chronic pelvic inflammatory disease and tubo-ovarian abscess formation. Septicemia, arthritis, endocarditis and meningitis are associated with complicated gonorrhea.

The high number of gonococcal strains with resistance to antibiotics contributes to increased morbidity and complications associated with gonorrhea. An attractive alternative to treatment of gonorrhea with antibiotics would be its prevention using vaccination. No vaccine currently exists for N. gonorrhoeae infections.

Neisseria meningitidis (meningococcus) is a Gram-negative bacterium frequently isolated from the human upper respiratory tract. It occasionally causes invasive bacterial diseases such as bacteremia and meningitis. Most cases of disease are in infants or young children. The incidence of meningococcal disease shows geographical seasonal and annual differences (Schwartz, B., Moore, P. S., Broome, C. V.; Clin. Microbiol. Rev. 2 (Supplement), S18-S24, 1989). Most disease in temperate countries is due to strains of serogroup B and varies in incidence from 1-10/100,000/year total population sometimes reaching higher values (Kaczmarski, E. B. (1997), Commun. Dis. Rep. Rev. 7: R55-9, 1995; Scholten, R. J. P. M., Bijlmer, H. A., Poolman, J. T. et al. Clin. Infect. Dis. 16: 237-246, 1993; Cruz, C., Pavez, G., Aguilar, E., et al. Epidemiol. Infect. 105: 119-126, 1990).

Epidemics dominated by serogroup A meningococci, mostly in central Africa, are encountered, sometimes reaching levels up to 1000/100.000/year (Schwartz, B., Moore, P. S., Broome, C. V. Clin. Microbiol. Rev. 2 (Supplement), S18-S24, 1989). Nearly all cases as a whole of meningococcal disease are caused by serogroup A, B, C, W-135 and Y meningococci and a tetravalent A, C, W-135, Y polysaccharide vaccine is available (Armand, J., Arminjon, F., Mynard, M. C., Lafaix, C., J. Biol. Stand. 10: 335-339, 1982).

The frequency of Neisseria meningitidis infections has risen dramatically in the past few decades. This has been attributed to the emergence of multiply antibiotic resistant strains and an increasing population of people with weakened immune systems. It is no longer uncommon to isolate Neisseria meningitidis strains that are resistant to some or all of the standard antibiotics. This phenomenon has created an unmet medical need and demand for new anti-microbial agents, vaccines, drug screening methods, and diagnostic tests for this organism.

Martin D et al Journal of Biotechnology 83 (2000) 27-31 reports that there is presently no effective vaccine that can stimulate protective group-common immunity in young children. Efforts are being made to improve the current polysaccharide vaccines through conjugation to carrier proteins or to find other meningococcal surface antigens that could become the basis of a protein vaccine. However, the interstrain variability of the major outer membrane proteins would restrict their protective efficacy to a limited number of antigenically related strains. Neisseria surface protein A (referred to herein as NspA) has characteristics which indicate that it is a potential vaccine candidate for the development of a group-common vaccine against meningocococcal disease.

It is envisaged that recombinant NspA expressed in cells could be produced for use in such new anti-microbial agents, vaccines, drug screening methods, and diagnostic tests. However, one of the major limitations on the expression of proteins is the inability of many recombinant proteins to fold into their biologically active conformations. Often only low yields of the recombinant protein are obtained due to aggregation and mis-folding of the unfolded species. Indeed, protein refolding, in which the protein acquires it native and active structure, is one of the biggest challenges in molecular biology.

Given the problems associated with obtaining biologically active refolded recombinant proteins, the use of non-live vectors, for example bacterial outer-membrane vesicles or “blebs” has been envisaged. OM blebs are derived from the outer membrane of the two-layer membrane of Gram-negative bacteria and have been documented in many Gram-negative bacteria (Zhou, L et al. 1998. FEMS Microbiol. Lett. 163:223-228). However, blebs have the disadvantage that they may express outer-membrane proteins which are either not relevant (e.g. unprotective antigens or immunodominant but variable proteins) or detrimental (e.g. toxic molecules such as LPS, or potential inducers of an autoimmune response). The need therefore remains to provide a subunit vaccine against Neisseria disease comprising purified protective outer membrane proteins in a refolded conformation suitable to elicit an effective immune response.

### SUMMARY OF THE INVENTION

The present invention provides an improved method for refolding the NspA protein. We have now shown that it is possible to increase the recovery of active protein from partly purified inclusion bodies in amounts up to 100%, without the need for further purification.

The present invention relates to refolded NspA protein and methods for using such proteins, including prevention and treatment of microbial diseases, amongst others in e.g. subunit vaccines. In a further aspect, the invention relates to diagnostic assays for detecting diseases associated with microbial infections and conditions associated with such infections.

### Statements of the Invention

According to one aspect of the present invention there is provide an isolated, refolded NspA protein (also referred to herein as “NspA”).

According to another aspect of the present invention there is provided a method for refolded NspA protein comprising contacting the NspA with an alkaline refolding buffer comprising 3-dimethyldodecylammoniopropanesulfonate (hereinafter referred to as SB-12).

Preferably the refolding buffer comprises ethanolamine and SB-12.

Preferably the refolding buffer has pH11.

Preferably the SB-12 is 0.2-1% or 0.3-0.8% SB-12.

Preferably the SB-12 is 0.2% SB-12.

In another preferred embodiment the SB-12 is 0.5% SB-12.

In further preferred embodiment the SB-12 is 1% SB-12.

Preferably the SB-12 is purified.

Preferably the SB-12 is purified by passing it over an Al₂O₃ column.

Preferably the ethanolamine is about 20 mM ethanolamine (most preferably about pH11).

According to another aspect of the present invention there is provided a method comprising any one of more (preferably all) of the following steps:
expressing NspA in a host cell;
breaking the host cell to obtain an inclusion body comprising NspA;
washing the inclusion body;
solubilisation of NspA and/or the inclusion body;
contacting the solubilised NspA with the refolding buffer; and
removing the refolding buffer from the NspA.

According to another aspect of the present invention there is provided a refolding buffer comprising ethanolamine and SB-12 (for instance in the concentrations mentioned above) for use in the method of the present invention.

According to another aspect of the present invention there is provided an isolated, refolded NspA protein obtained or obtainable by the method of the present invention.

According to another aspect of the present invention there is provided a pharmaceutical composition comprising at least one isolated, refolded NspA protein of the present invention, and a pharmaceutically acceptable carrier.

Preferably at least 30%, 50%, 70%, or 90% of the NspA protein present in the composition is refolded.

In one embodiment the pharmaceutical composition is in the form of a vaccine.

Preferably said composition comprises at least one other Neisserial antigen.

Preferably said composition further comprises at least one other Neisserial adhesin, or one other Neisserial outer membrane protein, or at least one Neisserial autotransporter, or at least one Neisserial toxin, or at least one Neisserial Fe acquisition protein. Most preferably, in addition to isolated, refolded NspA, at least one Neisserial autotransporter and at least one Neisserial toxin is present, or at least one Neisserial autotransporter and at least one Neisserial Fe acquisition protein is present, or at least one Neisserial toxin and at least one Neisserial Fe acquisition protein is present, or at least one Neisserial autotransporter and at least one Neisserial Fe acquisition protein and at least one Neisserial toxin is present, or immunogenic fragments thereof.

More preferably the pharmaceutical composition comprises at least one further antigen (or fragment thereof) selected from at least one of the following classes:
at least one Neisserial adhesin selected from the group consisting of FhaB, Hsf, NadA, PilC, Hap, MafA, MafB, Omp26, NMB0315, NMB0995 and NMB1119;
at least one Neisserial autotransporter selected from the group consisting of Hsf, Hap, IgA protease, AspA and NadA;
at least one Neisserial toxin selected from the group consisting of FrpA, FrpC, FrpA/C, VapD, NM-ADPRT, and either or both of LPS immunotype L2 and LPS immunotype L3;
at least one Neisserial Fe acquisition protein selected from the group consisting of TbpA high, TbpA low, TbpB high, TbpB low, LbpA, LbpB, P2086, HpuA, HpuB, Lipo28, Sibp, FbpA, BfrA, BfrB, Bcp, NMB0964 and NMB0293; and
at least one Neisserial membrane associated protein, preferably outer membrane protein, selected from the group consisting of PldA, TspA, FhaC, NspA, TbpA(high), TbpA(low), LbpA, HpuB, TdfH, PorB, HimD, HisD, GNA1870, OstA, H1pA, M1tA, NMB 1124, NMB 1162, NMB 1220, NMB 1313, NMB 1953, HtrA, TspB, Pi1Q and OMP85.

Most preferably the pharmaceutical composition comprises at least one further antigen (or fragment thereof) selected from at least one of the following classes:
at least one Neisserial adhesin selected from the group consisting of FhaB, Hsf and NadA;
at least one Neisserial autotransporter selected from the group consisting of Hsf, and Hap;
at least one Neisserial toxin selected from the group consisting of FrpA, FrpC, and either or both of LPS immunotype L2 and LPS immunotype L3;
at least one Neisserial Fe acquisition protein selected from the group consisting of TbpA, TbpB, LbpA and LbpB; and
at least one Neisserial outer membrane protein (or fragment thereof) selected from the group consisting of P1dA, TspA, TspB, Pi1Q and OMP85.

Preferably the pharmaceutical composition of the present invention comprises a further antigen derived from Neisseria meningitidis.

Preferably the pharmaceutical composition of the present invention further comprises an antigen derived from Neisseria gonorrhoeae.

Preferably the pharmaceutical composition of the present invention is at least in part a subunit preparation. Although it may be a subunit preparation mixed with a bleb preparation comprising additional Neisserial antigens of the invention (preferably upregulated in expression), it is also preferred that the pharmaceutical compositions of the present invention is entirely a subunit preparation with any additional Neisserial antigens of the invention being present in a refolded form, or as soluble surface-exposed fragment of the additional Neisserial antigen.

Preferably the pharmaceutical composition further comprises bacterial capsular polysaccharides or oligosaccharides.

Preferably said capsular polysaccharides or oligosaccharides are derived from bacteria selected from the group consisting of Neisseria meningitidis serogroup A, C, Y, and/or W-135, Haemophilus influenzae b, Streptococcus pneumoniae, Group A Streptococci, Group B Streptococci, Staphylococcus aureus and Staphylococcus epidermidis, and are most preferably conjugated to a source of T-helper epitopes.

According to another aspect of the present invention there is provided an antibody immunospecific for the NspA protein of the present invention.

According to another aspect of the present invention there is provided a method of diagnosing a Neisserial infection, comprising identifying an NspA protein of the present invention, or an antibody that is immunospecific for said protein, present within a biological sample from an animal, including a human, suspected of having such an infection, or by using an NspA protein or antibody of the present invention to detect whether NspA or antibodies against NspA are present within a biological sample from an animal.

Preferably the method relates to diagnosis of Neisseria meningitidis and most preferably Neisseria meningitidis serogroup B.

In another preferred embodiment the method relates to diagnosis of Neisseria gonorrhoeae.

According to another aspect of the present invention there is provided use of a composition comprising an NspA protein of the present invention in the preparation of a medicament for use in generating an immune response in an animal.

In one embodiment the use of the vaccine is in the preparation of a medicament for treatment or prevention of Neisserial infection.

In one preferred embodiment the use is in the preparation of a medicament for treatment or prevention of Neisseria meningitidis infection and most preferably Neisseria meningitidis serogroup B.

In another preferred embodiment the use is in the preparation of a medicament for the treatment or prevention of Neisseria gonorrhoeae infection.

According to yet another aspect of the present invention there is provided a pharmaceutical composition useful in treating humans with a Neisserial disease comprising at least one antibody directed against the NspA protein of the present invention and a suitable pharmaceutical carrier.

According to a further aspect of the present invention there is provided use of the antibody of the present invention in the manufacture of a medicament for the treatment or prevention of Neisserial disease.

According to one preferred embodiment Neisseria meningitidis infection is prevented or treated and most preferably Neisseria meningitidis serogroup B infection.

According to another preferred embodiment Neisseria gonorrhoeae infection is prevented or treated.

### DETAILED DESCRIPTION

Various preferred features and embodiments of the present invention will now be described by way of non-limiting example with reference to the accompanying drawing in which:

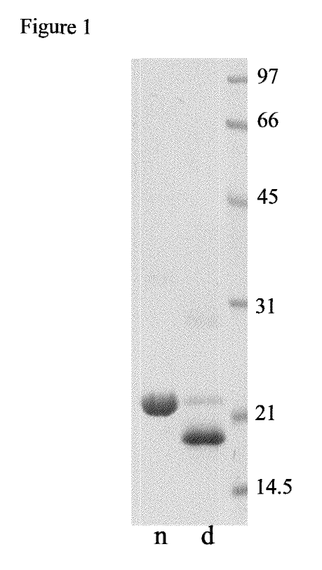

FIG. 1 shows an SDS-PAGE gel comparing NspA refolded according to the method of the present invention and denatured NspA. In more detail, FIG. 1 shows the heat-modifiability of purified refolded NspA in Coomassie Blue stained 14% PAGE. n=purified refolded NspA run in semi-native conditions, and d=purified refolded NspA run in denaturing conditions. The molecular weight markers are shown on the right.

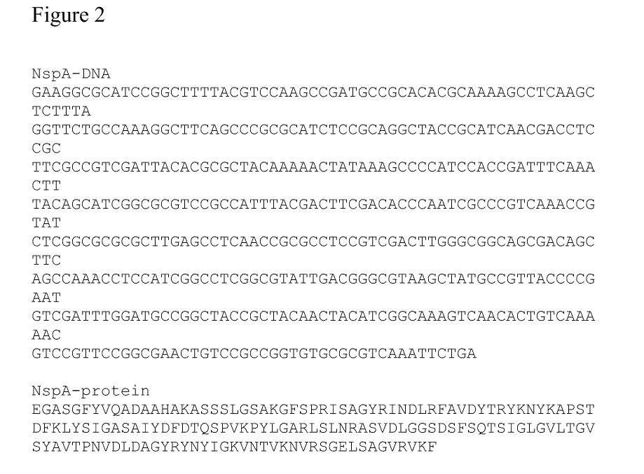

FIG. 2 shows the nucleotide (SEQ ID NO:5) and amino acid (SEQ ID NO:6) sequence of the H44/76 NspA used in the Examples; and

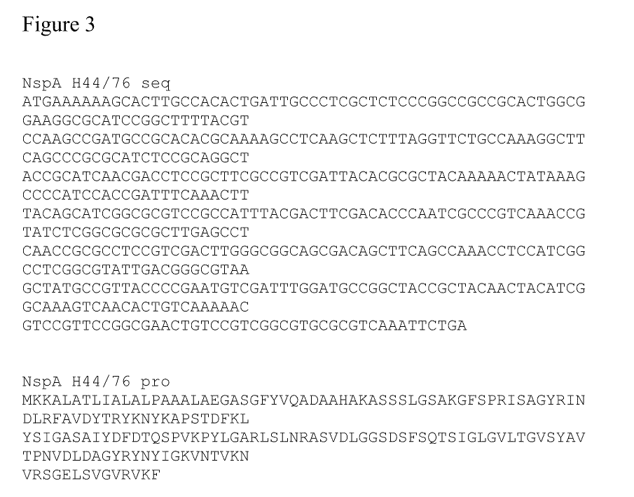

FIG. 3 shows the nucleotide (SEQ ID NO:7) and amino acid (SEQ ID NO:80 of NspA including leader sequence.

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc (as well as the complete version Current Protocols in Molecular Biology).

The terms “comprising”, “comprise” and “comprises” herein is intended by the inventors to be optionally substitutable with the terms “consisting of”, “consist of”, and “consists of”, respectively, in every instance.

### Method

The present invention provides a method for promoting the correct folding/refolding of an NspA protein which method involves the use of the detergent SB-12 in an alkaline refolding buffer.

Typically the method of the present invention is used to assist in refolding recombinantly produced NspA, which is obtained in an unfolded or misfolded form. Thus, recombinantly produced proteins may be contacted with the refolding buffer to unfold, refold and/or reactivate recombinant proteins which are inactive due to misfolding and/or are unfolded as a result of their extraction from the host cells in which they were expressed (such as from bacterial inclusion bodies). Such a process may also be termed “reconditioning”.

The method of the invention may be employed to maintain the folded conformation of NspA, for example during storage, in order to increase shelf life. Under storage conditions, many proteins lose their activity, as a result of disruption of correct folding. The presence of the refolding buffer of the present invention, reduces or reverses the tendency of proteins to become unfolded and thus greatly increases the shelf life thereof.

The method of the invention may be used to promote the correct folding of NspA which, through storage, exposure to denaturing conditions or otherwise, have become misfolded. Thus, the invention may be used to recondition NspA. For example, NspA in need of reconditioning may be contacted with the refolding buffer in accordance with the invention.

The present invention also provides a method for altering the structure of an NspA protein. Structural alterations include folding, unfolding and refolding. The effect of the alterations is preferably to improve the yield, specific activity and/or quality of the molecule. This may typically be achieved by resolubilising, reconditioning and/or reactivating incorrectly folded molecules post-synthesis.

The terms “reconditioning” and “reactivating” thus encompass in vitro procedures. Particular examples of in vitro procedures may include processing proteins that have been solubilised from cell extracts (such as inclusion bodies) using strong denaturants such as urea or guanidium chloride.

The terms “refold”, “reactivate” and “recondition” are not intended as being mutually exclusive. For example, an inactive protein, perhaps denatured using urea, may have an unfolded structure. This inactive protein may then be refolded with a refolding buffer of the invention thereby reactivating it. In some circumstances there may be an increase in the specific activity of the refolded/reactivated protein compared to the protein prior to inactivation/denaturation: this is termed “reconditioning”.

The molecule is typically an unfolded or misfolded protein which is in need of folding. Alternatively, however, it may be a folded protein which is to be maintained in a folded state.

The invention envisages at least two situations. A first situation is one in which the protein to be folded is in an unfolded or misfolded state, or both. In this case, its correct folding is promoted by the method of the invention. A second situation is one in which the protein is substantially already in its correctly folded state, that is all or most of it is folded correctly or nearly correctly. In this case, the method of the invention serves to maintain the folded state of the protein by affecting the folded/unfolded equilibrium so as to favour the folded state. This prevents loss of activity of an already substantially correctly folded protein. These, and other, eventualities are covered by the reference to “promoting” the folding of the protein.

As used herein, a protein may be unfolded when at least part of it has not yet acquired its correct or desired secondary or tertiary structure. A protein is misfolded when it has acquired at least partially incorrect or undesired secondary or tertiary structure. Techniques are known in the art for assessing protein structure—such as circular dichroism.

The refolding buffer of the present invention comprises 3-dimethyldodecylammoniopropanesulfonate (also referred to as N-Dodecyl-N,N-dimethyl-3-ammonio-1-propanesulfonate) and referred to herein as “SB-12”. SB-12 is available from commercial sources, such as Fluka AG. Although SB-12 is highly preferred, the use of other salts of SB12 may also be used, as well as derivatives of SB-12 or molecules related to SB-12. Whilst not wishing to be bound by any theory, we believe the dilution step in SB-12 (or the presence of SB-12 in general) creates a hydrophobic environment for the protein, which is similar to the protein's natural in vivo environment.

SB-12 is a detergent and the concentration of SB-12 should be at least about 0.2% (w/v), since this is the concentration generally required for micelle formation. Thus, the concentration of SB-12 may be about 0.2% to about 5.0%, about 0.3% to about 4.0%, about 0.4% to about 3.0%, or about 0.5% to about 2.0%. Preferably the concentration is about 0.2%, 0.3%, 0.4%, 0.5%, 0.6%, 0.7%, 0.8%, 0.9% or 1.0%. In an especially preferred embodiment 0.5% SB-12 is used. By “about” it is preferably meant+/−10% of the value of the figure quoted, but is most preferably the exact figure quoted.

In a preferred embodiment the SB-12 is purified. We have found improved folding with purified SB-12 compared to non-purified SB-12. Conveniently the SB-12 may be purified before use by passing a concentrated solution of SB-12 over an Al₂O₃ chromatography column, e.g. using in methanol/chloroform (1:1), but any suitable method may be used for purifying the SB-12.

We have found that the dilution of the denatured protein should to be carried out in an alkaline environment to maximise the efficiency of refolding. Preferably the pH of the refolding buffer is about 11.0. Preferably the alkaline environment is obtained by the use of ethanolamine. In this preferred embodiment the refolding buffer comprises SB-12 and ethanolamine. Conveniently 20 mM ethanolamine is used, but other concentrations, such as 50 mM, may be useful.

We have found that a 1:20 dilution of the NspA in the refolding buffer is preferred, but other ratios such as 1:10 may be used. Five-fold dilution into refolding buffer may also be used.

The NspA to be processed by the method of the invention is typically obtained from cell extracts of host cells expressing recombinant NspA. Host cells include prokaryotes such as E. coli, yeast and insect cells (the baculovirus system is capable of very high level protein expression). Expression of the NspA in the host cell is preferably at high levels to maximise yield. Further details on the expression of recombinant NspA are given below.

We have found that the present invention is most efficient when used to refold a mature NspA protein, i.e. a protein without a leader or secretory sequence, a pre-, or pro- or prepro-protein sequence, and also a protein. Typically, for NspA, this means expressing the protein without the signal peptide (amino acids 1-19). It is common during conventional purification techniques to make use of a marker sequence that facilitates purification, such as a hexa-histidine peptide, as provided in the pQE vector (Qiagen, Inc.) and described in Gentz et al., Proc. Natl. Acad. Sci., USA 86: 821-824 (1989), or an HA peptide tag (Wilson et al., Cell 37: 767 (1984). We have found that the process of the present invention is most efficient when such marker sequences are not present.

It is likely that a substantial proportion of the NspA will be insoluble and consequently techniques to solubilise normally insoluble components of the cell extracts (such as inclusion bodies) to maximise extraction of the NspA will typically be employed. Any conventional technique for preparation and extraction of the NspA proteins from inclusion bodies and their subsequent solubilisation may be employed. Such techniques are described for example in “Current Protocols in Protein Science” published by JA Wiley & Sons. Such techniques generally include:
Cell Lysis—using, for example, a French press or sonication, to release inclusion bodies. Typically the cells are placed in a cold buffer such as a TE buffer prior to lysis. Sonication may be carried out using a Branson sonifier. Sonification may take place in the presence of a detergent, e.g. BRIJ™ or TRITON™. The inclusion bodies in the cell lysate may then be pelleted using low-speed centrifugation. The cells may be pretreated with lysozyme prior to lysis. The purpose of the pretreatment is to aid removal of the peptidoglycan and outer membrane protein contaminants during the washing steps. The lysed cells may be clarified by centrifugation and the supernatant discarded.
Inclusion Body Washing—to remove cell wall and other outer membrane components contaminants from the inclusion bodies recovered from cell lysates. Typically the pellet is resuspended in a wash buffer containing, e.g. buffer such as TE buffer and/or a detergent such as TRITON™. The suspension may then be resuspended and the supernatant discarded. This process may be repeated until the supernatant is clear. If required, the washed pellets can be frozen for storage.

The amount of recombinant protein in the washed pellet may be estimated using the following guidelines: (1) an expression level of 1% corresponds to ˜1 mg recombinant protein per 1 g wet cells. (2) The recovery of highly aggregated recombinant protein in the washed pellets is ˜75% that originally present in the cells. (3) About 60% of the total washed pellet protein is recombinant-derived. The total amount of recombinant protein can be directly determined be measuring the total protein concentration or by analysing the washed pellets via SDS-PAGE to determine the proportions of the protein constituents.

Protein solubilisation—the extracted protein is then extracted from the washed pellet and defolded using a denaturant which disassociates protein-protein interactions and unfolds the protein so that it consists of unfolded monomers. Denaturants include guanidine:HC1 (such as 6M guanidine:HC1) and/or urea (such as 8M urea). Residual insoluble material and materials can be typically removed by ultracentrifugation (e.g. 100,000 g×for 1 hr.). The extract may be stored by freezing following pelleting.

Solubilised cell extracts may optionally be partially purified by, for example, a variety of affinity chromatography techniques prior to contacting with the refolding buffer according to the method of the invention.

The solubilised cell extract is then diluted into the refolding buffer in accordance with the present invention. It is typically, but not necessarily, left stirring at room temperature overnight.

Thus, the starting material for the refolding/reconditioning method of the invention is typically denatured proteins in solutions of agents such as urea/guanidium chloride. Alternatively, or in addition, soluble protein samples may be specifically denatured by the addition of appropriate denaturing agents prior to refolding.

The method of the invention may also employ the use of molecular chaperones. Chaperones, including chaperonins, are proteins which promote protein folding by non-enzymatic means, in that they do not catalyse the chemical modification of any structures in folding proteins, but promote the correct folding of proteins by facilitating correct structural alignment thereof. Molecular chaperones are well known in the art, several families thereof being characterised. The invention may employ any molecular chaperone molecule, which term includes, for example, the molecular chaperones selected from the following non-exhaustive group:

p90 Calnexin, HSP family, HSP70 family, DNA K, DNAJ, HSP60 family (GroEL), ER-associated chaperones, HSP90, Hsc70, sHsps; SecA; SecB, Trigger factor, zebrafish hsp 47, 70 and 90, HSP 47, GRP 94, Cpn 10, BiP, GRP 78, Clp, FtsH, Ig invariant chain, mitochondrial hsp70, EBP, mitochondrial m-AAA, Yeast Ydj 1, Hsp104, ApoE, Syc, Hip, TriC family, CCT, PapD and calmodulin (see WO99/05163 for references).

The method of the present invention may also make use of a foldase. In general terms, a foldase is an enzyme which participates in the promotion of protein folding through its enzymatic activity to catalyse the rearrangement or isomerisation of bonds in the folding protein. They are thus distinct from a molecular chaperone, which bind to proteins in unstable or non-native structural states and promote correct folding without enzymatic catalysis of bond rearrangement. Many classes of foldase are known, and they are common to animals, plants and bacteria. They include peptidyl prolyl isomerases and thiol/disulphide oxidoreductases. The invention may employ any of the foldases which are capable of promoting protein folding through covalent bond rearrangement.

At the end of the refolding/reconditioning process, the refolded NspA may be desalted by dialysis against a suitable storage buffer and/or the use of a desalting column into a suitable storage buffer. Suitable buffers include 25 mM sodium phosphate, 150 mM NaCl and 0.1% PEG 6000 (pH 7.4).

### Vectors, Host Cells, Expression Systems

The invention may employ vectors that comprise a polynucleotide which codes for at least an NspA protein, host cells that are genetically engineered with vectors of the invention and the production of NspA proteins by recombinant techniques. Cell-free translation systems can also be employed to produce such proteins using RNAs derived from DNA constructs.

Recombinant proteins of the present invention may be prepared by processes well known to those skilled in the art from genetically engineered host cells comprising expression systems.

For recombinant production of the proteins of the invention, host cells can be genetically engineered to incorporate expression systems or portions thereof or polynucleotides of the invention. Introduction of a polynucleotide into the host cell can be effected by methods described in many standard laboratory manuals, such as Davis, et al., BASIC METHODS IN MOLECULAR BIOLOGY, (1986) and Sambrook, et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as cells of streptococci, staphylococci, enterococci, E. coli, streptomyces, cyanobacteria, Bacillus subtilis, Moraxella catarrhalis, Haemophilus influenzae and Neisseria meningitidis; fungal cells, such as cells of a yeast, Kluveromyces, Saccharomyces, a basidiomycete, Candida albicans and Aspergillus; insect cells such as cells of Drosophila S2 and Spodoptera Sf9; animal cells such as CHO, COS, HeLa, C127, 3T3, BHK, 293, CV-1 and Bowes melanoma cells; and plant cells, such as cells of a gymnosperm or angiosperm.

A great variety of expression systems can be used to produce the proteins of the invention. Such vectors include, among others, chromosomal-, episomal- and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, picornaviruses, retroviruses, and alphaviruses and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a protein in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

In recombinant expression systems in eukaryotes, for secretion of a translated protein into the lumen of the endoplasmic reticulum, into the periplasmic space or into the extracellular environment, appropriate secretion signals may be incorporated into the expressed protein. These signals may be endogenous to the protein or they may be heterologous signals.

Proteins of the present invention can be recovered and purified from recombinant cell cultures by the method of the present invention.

NspA protein

The present invention provides an isolated, refolded NspA protein. As used herein the term “protein” includes the term “polypeptide”. By “isolated” we mean that the NspA protein is free from other proteins with which it is normal associated. The NspA protein may be a recombinant protein. By “recombinant” we mean that the protein has been obtained using the application of molecular biology. However the method is also applicable to natural or synthetic proteins which require refolding, or purification by means of unfolding and refolding. Preferably the isolated, refolded NspA of the invention is purified in that it is more than 40, 50, 60, 70, 80, 90, 95, or 99% pure. Most preferably the NspA of the invention is biologically pure in that it is more than 40, 50, 60, 70, 80, 90, 95, or 99% free of other Neisserial proteins and/or other proteins of the host cell from which it was made.

Where a protein is specifically mentioned herein, it is preferably a reference to a native, full-length protein but it may also encompass antigenic fragments thereof (particularly in the context of subunit vaccines). These are fragments which contain or comprise at least 10 amino acids, preferably 20 amino acids, more preferably 30 amino acids, more preferably 40 amino acids or most preferably 50 amino acids, taken contiguously from the amino acid sequence of the protein. In addition, antigenic fragments denotes fragments that are immunologically reactive with antibodies generated against the Neisserial proteins or with antibodies generated by infection of a mammalian host with Neisseria. Antigenic fragments also includes fragments that when administered at an effective dose, elicit a protective immune response against Neisserial infection, more preferably it is protective against N. meningitidis and/or N. gonorrhoeae infection, most preferably it is protective against N. meningitidis serogroup B infection.

The present invention also includes variants of the proteins mentioned herein, that is proteins that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

It has been reported that the highly conserved NspA protein has been found in the outer membrane of every Neisseria meningitidis strain tested so far (Martin D et al Journal of Biotechnology 83 (2000) 27-31). Martin D et al also show that the NspA protein is exposed at the surface of intact meningococcal strains. The NspA protein has also been identified in Neisseria gonorrhoeae (Plante et al Infect. Immun. 67 (1999) 2855-2861). The meningococcal nspA gene may be amplified directly by PCR from chromosomal DNA (Martin D et al & Plante et al 1999 supra). NspA is also described in WO96/29412.

The NspA produced by the present invention, fragment or variant thereof, preferably is a product which displays the immunological activity of the wild type NspA protein (for instance as present on isolated Neisserial blebs). Preferably it will show at least one of the follow: An ability to induce the production of antibodies which recognise the wild type NspA (if necessary when the NspA protein of the present invention is coupled to a carrier); An ability to induce the production of antibodies that can protect against experimental infection; and

An ability to induce, when administered to an animal, the development of an immunological response that can protect against Neisserial infection such as Neisseria meningitidis and/or Neisseria gonorrhoeae infection.

The NspA protein of the present invention is useful in prophylactic, therapeutic and diagnostic compositions for preventing treating and diagnosing diseases caused by Neisseria, particularly Neisseria meningitidis; although it may also have similar applications in relation to, e.g. Neisseria gonorrhoeae or Neisseria lactamica.

Standard immunological techniques may be employed with the NspA protein of the present invention in order to use it as an immunogen and in a vaccine. In particular, any suitable host may be injected with a pharmaceutically effective amount of the NspA protein to generate monoclonal or polyclonal anti-NspA antibodies or to induce the development of a protective immunological response against a Neisseria disease. Prior to administration, the NspA protein may be formulated in a suitable vehicle, and thus we provide a pharmaceutical composition comprising a pharmaceutically effective amount of one or more proteins of the present invention. As used herein “pharmaceutically effective amount” refers to an amount of NspA protein (or other proteins of the invention) that elicits a sufficient titre of antibodies to treat or prevent infection. The pharmaceutical composition of the present invention may also comprise other antigens useful in treating or preventing disease.

The NspA protein of this invention may also form the basis of a diagnostic test for infection. For example, the present invention provides a method for detection of a Neisserial antigen in a biological sample containing or suspected of containing the Neisserial antigen comprising:
generating an anti-NspA antibody using the protein of the present invention;
isolating the biological sample from a patient;
incubating the anti-NspA antibody or a fragment thereof with the biological sample; and
detecting bound antibody or bound fragment.

This invention also provides a method for the detection of antibody specific to NspA protein in a biological sample containing or suspected of containing said antibody comprising:
isolating the biological sample from a patient;
incubating the NspA protein of the invention with the biological sample; and
detecting bound antigen.

This diagnostic test may take several forms including ELISA and a radioimmunoassay.

Further details on such applications are given below.

### Antibodies

The proteins of the invention can be used as immunogens to produce antibodies immunospecific for such proteins.

In certain preferred embodiments of the invention there are provided antibodies against the NspA protein of the invention.

Antibodies generated against the proteins of the invention can be obtained by administering the proteins of the invention, or epitope-bearing fragments of either or both, analogues of either or both, to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985).

Techniques for the production of single chain antibodies (U.S. Pat. No. 4,946,778) can be adapted to produce single chain antibodies to proteins of this invention. Also, transgenic mice, or other organisms or animals, such as other mammals, may be used to express humanized antibodies immunospecific to the proteins of the invention.

Alternatively, phage display technology may be utilized to select antibody genes with binding activities towards a protein of the invention either from repertoires of PCR amplified v-genes of lymphocytes from humans screened for possessing anti-NspA or from naive libraries (McCafferty, et al., (1990), Nature 348, 552-554; Marks, et al., (1992) Biotechnology 10, 779-783). The affinity of these antibodies can also be improved by, for example, chain shuffling (Clackson et al., (1991) Nature 352: 628).

The above-described antibodies may be employed to isolate or to identify clones expressing NspA proteins of the invention to purify the proteins or polynucleotides by, for example, affinity chromatography.

Thus, among others, antibodies against the NspA protein of the inventon may be employed to treat infections, particularly bacterial infections.

Preferably, the antibody or variant thereof is modified to make it less immunogenic in the individual. For example, if the individual is human the antibody may most preferably be “humanized,” where the complimentarity determining region or regions of the hybridoma-derived antibody has been transplanted into a human monoclonal antibody, for example as described in Jones et al. (1986), Nature 321, 522-525 or Tempest et al., (1991) Biotechnology 9, 266-273.

A protein of the present invention can be administered to a recipient who then acts as a source of immune globulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat Neisserial infection. Hyperimmune globulins of the invention are particularly useful for treatment or prevention of Neisserial disease in infants, immune compromised individuals or where treatment is required and there is no time for the individual to produce antibodies in response to vaccination.

An additional aspect of the invention is a pharmaceutical composition comprising a monoclonal antibody (or fragments thereof; preferably human or humanised) reactive against the pharmaceutical composition of the invention, which could be used to treat or prevent infection by Gram negative bacteria, preferably Neisseria, more preferably Neisseria meningitidis or Neisseria gonorrhoeae and most preferably Neisseria meningitidis serogroup B.

Such pharmaceutical compositions comprise monoclonal antibodies that can be whole immunoglobulins of any class e.g. IgG, IgM, IgA, IgD or IgE, chimeric antibodies or hybrid antibodies with specificity to two or more antigens of the invention. They may also be fragments e.g. F(ab′)₂, Fab′, Fab, Fv and the like including hybrid fragments.

Methods of making monoclonal antibodies are well known in the art and can include the fusion of splenocytes with myeloma cells (Kohler and Milstein 1975 Nature 256; 495; Antibodies—a laboratory manual Harlow and Lane 1988). Alternatively, monoclonal Fv fragments can be obtained by screening a suitable phage display library (Vaughan T J et al 1998 Nature Biotechnology 16; 535). Monoclonal antibodies may be humanised or part humanised by known methods.

### Antagonists and Agonists-Assays and Molecules

The refolded proteins of the invention may also be used to assess the binding of small molecule substrates and ligands in, for example, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

The screening methods may simply measure the binding of a candidate compound to the protein. Alternatively, the screening method may involve competition with a labeled competitor. The screening methods may simply comprise the steps of mixing a candidate compound with a solution containing a protein of the present invention, to form a mixture, measuring NspA protein activity in the mixture, and comparing the NspA protein activity of the mixture to a standard.

The polynucleotides, proteins and antibodies that bind to and/or interact with a protein of the present invention may also be used to configure screening methods for detecting the effect of added compounds on the production of mRNA and/or protein in cells. For example, an ELISA assay may be constructed for measuring secreted or cell associated levels of protein using monoclonal and polyclonal antibodies by standard methods known in the art. This can be used to discover agents which may inhibit or enhance the production of protein (also called antagonist or agonist, respectively) from suitably manipulated cells or tissues.

The invention also provides a method of screening compounds to identify those which enhance (agonist) or block (antagonist) the action of NspA proteins, particularly those compounds that are bacteristatic and/or bactericidal. The method of screening may involve high-throughput techniques. For example, to screen for agonists or antagonists, a synthetic reaction mix comprising NspA protein and a labeled substrate or ligand of such protein is incubated in the absence or the presence of a candidate molecule that may be a NspA agonist or antagonist. The ability of the candidate molecule to agonize or antagonize the NspA protein is reflected in decreased binding of the labeled ligand or decreased production of product from such substrate. Molecules that bind gratuitously, i.e., without inducing the effects of NspA protein are most likely to be good antagonists. Reporter systems that may be useful in this regard include but are not limited to colorimetric, labeled substrate converted into product, a reporter gene that is responsive to changes in NspA protein activity, and binding assays known in the art.

Another example of an assay for NspA agonists is a competitive assay that combines NspA and a potential agonist with NspA-binding molecules, recombinant NspA binding molecules, natural substrates or ligands, or substrate or ligand mimetics, under appropriate conditions for a competitive inhibition assay. NspA can be labeled, such as by radioactivity or a colorimetric compound, such that the number of NspA molecules bound to a binding molecule or converted to product can be determined accurately to assess the effectiveness of the potential antagonist.

Potential antagonists include, among others, small organic molecules, peptides, proteins and antibodies that bind to a polynucleotide and/or protein of the invention and thereby inhibit or extinguish its activity or expression. Potential antagonists also may be small organic molecules, a peptide, a protein such as a closely related protein or antibody that binds the same sites on a binding molecule, such as a binding molecule.

Potential antagonists include a small molecule that binds to and occupies the binding site of the protein thereby preventing binding to cellular binding molecules, such that normal biological activity is prevented. Examples of small molecules include but are not limited to small organic molecules, peptides or peptide-like molecules. Other potential antagonists include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules). Preferred potential antagonists include compounds related to and variants of NspA.

The invention also provides the use of the protein, agonist or antagonist of the invention to interfere with the initial physical interaction between a pathogen or pathogens and a eukaryotic, preferably mammalian, host responsible for sequelae of infection. In particular, the molecules of the invention may be used: in the prevention of adhesion of bacteria, in particular gram positive and/or gram negative bacteria, to eukaryotic, preferably mammalian, extracellular matrix proteins on in-dwelling devices or to extracellular matrix proteins in wounds; to block bacterial adhesion between eukaryotic, preferably mammalian, extracellular matrix proteins and bacterial NspA proteins that mediate tissue damage and/or; to block the normal progression of pathogenesis in infections initiated other than by the implantation of in-dwelling devices or by other surgical techniques.

In accordance with yet another aspect of the invention, there are provided NspA agonists and antagonists, preferably bacteristatic or bactericidal agonists and antagonists.

The antagonists and agonists of the invention may be employed, for instance, to prevent, inhibit and/or treat diseases.

### Vaccines

Another aspect of the invention relates to a method for inducing an immunological response in an individual, particularly a mammal, preferably humans, which comprises inoculating the individual with NspA protein of the present invention, or a fragment or variant thereof, adequate to produce antibody and/or T cell immune response to protect (or treat) said individual from infection, particularly bacterial infection and most particularly Neisseria meningitidis infection. Also provided are methods whereby such immunological response slows bacterial replication.

A further aspect of the invention relates to an immunological composition that when introduced into an individual, preferably a human, capable of having induced within it an immunological response, induces an immunological response in such individual to a NspA protein of the present invention, wherein the composition comprises a recombinant NspA of the invention. The immunological response may be used therapeutically or prophylactically and may take the form of antibody immunity and/or cellular immunity, such as cellular immunity arising from CTL or CD4+T cells.

A NspA protein, variant or a fragment thereof may be fused with co-protein or chemical moiety which may or may not by itself produce antibodies, but which is capable of producing a fused or modified protein which will have antigenic and/or immunogenic properties, and preferably protective properties, and optionally may stabilise the NspA, or render it easier to purify. The co-protein may act as an adjuvant in the sense of providing a generalized stimulation of the immune system of the organism receiving the protein.

Also provided by this invention are compositions, particularly vaccine compositions, and methods comprising the proteins of the invention and immunostimulatory DNA sequences, such as those described in Sato, Y. et al. Science 273: 352 (1996).

The invention thus also includes a vaccine formulation which comprises an immunogenic protein of the invention or a fragment or a variant thereof, together with a suitable carrier, such as a pharmaceutically acceptable carrier. Since the proteins may be broken down in the stomach, each is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteristatic compounds and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The formulation may also be administered mucosally, e.g. intranasally.

The vaccine formulation of the invention may also include adjuvant systems for enhancing the immunogenicity of the formulation. Typically aluminium phosphate or aluminium hydroxide may be used. Preferably the adjuvant system raises preferentially a TH1 type of response.

An immune response may be broadly distinguished into two extreme categories, being a humoral or cell mediated immune responses (traditionally characterised by antibody and cellular effector mechanisms of protection respectively). These categories of response have been termed TH1-type responses (cell-mediated response), and TH2-type immune responses (humoral response).

Extreme TH1-type immune responses may be characterised by the generation of antigen specific, haplotype restricted cytotoxic T lymphocytes, and natural killer cell responses. In mice TH1-type responses are often characterised by the generation of antibodies of the IgG2a subtype, whilst in the human these correspond to IgG1 type antibodies. TH2-type immune responses are characterised by the generation of a broad range of immunoglobulin isotypes including in mice IgG1, IgA, and IgM.

It can be considered that the driving force behind the development of these two types of immune responses are cytokines. High levels of TH1-type cytokines tend to favour the induction of cell mediated immune responses to the given antigen, whilst high levels of TH2-type cytokines tend to favour the induction of humoral immune responses to the antigen.

The distinction of TH1 and TH2-type immune responses is not absolute. In reality an individual will support an immune response which is described as being predominantly TH1 or predominantly TH2. However, it is often convenient to consider the families of cytokines in terms of that described in murine CD4 +ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p145-173). Traditionally, TH1-type responses are associated with the production of the INF-γ and IL-2 cytokines by T-lymphocytes. Other cytokines often directly associated with the induction of TH1-type immune responses are not produced by T-cells, such as IL-12. In contrast, TH2-type responses are associated with the secretion of IL-4, IL-5, IL-6 and IL-13.

It is known that certain vaccine adjuvants are particularly suited to the stimulation of either TH1 or TH2-type cytokine responses. Traditionally the best indicators of the TH1:TH2 balance of the immune response after a vaccination or infection includes direct measurement of the production of TH1 or TH2 cytokines by T lymphocytes in vitro after restimulation with antigen, and/or the measurement of the IgG1:IgG2a ratio of antigen specific antibody responses.

Thus, a TH1-type adjuvant is one which preferentially stimulates isolated T-cell populations to produce high levels of TH1-type cytokines when re-stimulated with antigen in vitro, and promotes development of both CD8+cytotoxic T lymphocytes and antigen specific immunoglobulin responses associated with TH1-type isotype.

Adjuvants which are capable of preferential stimulation of the TH1 cell response are described in International Patent Application No. WO 94/00153 and WO 95/17209.

3 De-O-acylated monophosphoryl lipid A (3D-MPL) is one such adjuvant, and is preferred. This is known from GB 2220211 (Ribi). Chemically it is a mixture of 3 De-O-acylated monophosphoryl lipid A with 4, 5 or 6 acylated chains and is manufactured by Ribi Immunochem, Montana. A preferred form of 3 De-O-acylated monophosphoryl lipid A is disclosed in European Patent 0 689 454 B1 (SmithKline Beecham Biologicals SA). Alternatively, other non-toxic derivatives of LPS may be used.

Preferably, the particles of 3D-MPL are small enough to be sterile filtered through a 0.22 micron membrane (European Patent number 0 689 454). 3D-MPL will be present in the range of 10 μg-100 μg preferably 25-50 μg per dose wherein the antigen will typically be present in a range 2-50 μg per dose.

Another preferred adjuvant comprises QS21, an Hplc purified non-toxic fraction derived from the bark of Quillaja Saponaria Molina. Optionally this may be admixed with 3 De-O-acylated monophosphoryl lipid A (3D-MPL), or non-toxic LPS derivative, optionally together with a carrier.

The method of production of QS21 is disclosed in U.S. Pat. No. 5,057,540.

Non-reactogenic adjuvant formulations containing QS21 have been described previously (WO 96/33739). Such formulations comprising QS21 and cholesterol have been shown to be successful TH1 stimulating adjuvants when formulated together with an antigen.

Further adjuvants which are preferential stimulators of TH1 cell response include immunomodulatory oligonucleotides, for example unmethylated CpG sequences as disclosed in WO 96/02555.

Combinations of different TH1 stimulating adjuvants, such as those mentioned hereinabove, are also contemplated as providing an adjuvant which is a preferential stimulator of TH1 cell response. For example, QS21 can be formulated together with 3D-MPL. The ratio of QS21:3D-MPL will typically be in the order of 1:10 to 10:1; preferably 1:5 to 5:1 and often substantially 1:1. The preferred range for optimal synergy is 2.5:1 to 1:1 3D-MPL: QS21.

Preferably a carrier is also present in the vaccine composition according to the invention. The carrier may be an oil in water emulsion, or an aluminium salt, such as aluminium phosphate or aluminium hydroxide.

A preferred oil-in-water emulsion comprises a metabolisible oil, such as squalene, alpha tocopherol and TWEEN 80™. In a particularly preferred aspect the antigens in the vaccine composition according to the invention are combined with QS21 and 3D-MPL in such an emulsion. Additionally the oil in water emulsion may contain span 85 and/or lecithin and/or tricaprylin.

Typically for human administration QS21 and 3D-MPL will be present in a vaccine in the range of 1 μg-200 μg, such as 10-100 μg, preferably 10 μg-50 μg per dose. Typically the oil in water will comprise from 2 to 10% squalene, from 2 to 10% alpha tocopherol and from 0.3 to 3% TWEEN 80™. Preferably the ratio of squalene: alpha tocopherol is equal to or less than 1 as this provides a more stable emulsion. Span 85 may also be present at a level of 1%. In some cases it may be advantageous that the vaccines of the present invention will further contain a stabiliser.

Non-toxic oil in water emulsions preferably contain a non-toxic oil, e.g. squalane or squalene, an emulsifier, e.g. TWEEN 80™, in an aqueous carrier. The aqueous carrier may be, for example, phosphate buffered saline.

A particularly potent adjuvant formulation involving QS21, 3D-MPL and tocopherol in an oil in water emulsion is described in WO 95/17210.

The present invention also provides a polyvalent vaccine composition comprising a vaccine formulation of the invention in combination with other antigens, in particular antigens useful for treating cancers, autoimmune diseases and related conditions. Such a polyvalent vaccine composition may include a TH-1 inducing adjuvant as hereinbefore described.

### Subunit Composition

The composition of the present invention is preferably in the form of a subunit composition. Subunit compositions are compositions in which the components have been isolated and purified to at least 50%, preferably at least 60%, 70%, 80%, 90% pure before mixing the components to form the antigenic composition.

Subunit compositions may comprise aqueous solutions of water soluble proteins. They may comprise detergent, preferably non-ionic, zwitterionic or ionic detergent in order to solubilise hydrophobic portions of the antigens. They may comprise lipids so that liposome structures could be formed, allowing presentation of antigens with a structure that spans a lipid membrane. Further details on compositions is given below.

The subunit composition of the invention may also comprise at least one further antigen which may be soluble, or may be presented on the outer membrane of a bleb (OMV) (thus making a mixed subunit/bleb composition).

Neisserial infections progress through several different stages. For example, the meningococcal life cycle involve nasopharyngeal colonisation, mucosal attachment, crossing into the bloodstream, multiplication in the blood, induction of toxic shock, crossing the blood/brain barrier and multiplication in the cerebrospinal fluid and/or the meninges. Different molecules on the surface of the bacterium will be involved in different steps of the infection cycle. By targeting the immune response against an effective amount of a combination of particular antigens, involved in different processes of Neisserial infection, a Neisserial vaccine with surprisingly high efficacy can be achieved.

In particular, combinations of certain Neisserial antigens from different classes with the NspA protein of the invention can elicit an immune response which protects against multiple stages of infection. Such combinations of antigens can surprisingly lead to synergistically improved vaccine efficacy against Neisserial infection where more than one function of the bacterium is targeted by the immune response in an optimal fashion. Some of the further antigens which can be included are involved in adhesion to host cells, some are involved in iron acquisition, some are autotransporters and some are toxins. As NspA is an adhesin, it is preferred that it is mixed with an iron acquisition protein, autotransporter or toxin, or an iron acquisition protein and autotransporter, or an iron acquisition protein and a toxin, or an autotransporter and a toxin, or an iron acquisition protein and autotransporter and toxin.

The efficacy of vaccines can be assessed through a variety of assays. Protection assays in animal models are well known in the art. Furthermore, serum bactericidal assay (SBA) is the most commonly agreed immunological marker to estimate the efficacy of a meningococcal vaccine (Perkins et al. J Infect Dis. 1998, 177:683-691).

Some combinations of antigens can lead to improved protection in animal model assays and/or synergistically higher SBA titres. Without wishing to be bound by theory, such synergistic combinations of antigens are enabled by a number of characteristics of the immune response to the antigen combination. The antigens themselves are usually surface exposed on the Neisserial cells and tend to be conserved but also tend not to be present in sufficient quantity on the surface cell for an optimal bactericidal response to take place using antibodies elicited against the antigen alone. Combining the antigens of the invention can result in a formulation eliciting an advantageous combination of bactericidal antibodies which interact with the Neisserial cell beyond a critical threshold. At this critical level, sufficient antibodies of sufficient quality bind to the surface of the bacterium to allow efficient killing by complement and much higher bactericidal effects are seen as a consequence. As serum bactericidal assays (SBA) closely reflect the efficacy of vaccine candidates, the attainment of good SBA titres by a combination of antigens is a good indication of the protective efficacy of a vaccine containing that combination of antigens.

An additional advantage of the invention is that the combination of the antigens of the invention from different families of proteins in an immunogenic composition will enable protection against a wider range of strains.

The present invention also relates to a combination of NspA of the invention with one or more (e.g. two, three, four, five, six, seven, eight, nine or ten) different Neisserial proteins. Such additional proteins may be selected from the group consisting of: FhaB, Hsf, NadA, Hap, FrpA, FrpB, FrpC, LPS immunotype L2, LPS immunotype L3, TbpA, TbpB, LbpA, LbpB, TspA, TspB, PilQ, omp85 and P1dA.

The invention also relates to immunogenic compositions comprising a plurality of proteins selected from at least two different categories of protein, having different functions within Neisseria. Examples of such categories of proteins are adhesins, autotransporter proteins, toxins and Fe acquisition proteins. The vaccine combinations of the invention show surprising improvement in vaccine efficacy against homologous Neisserial strains (strains from which the antigens are derived) and preferably also against heterologous Neisserial strains.

In particular, the invention provides immunogenic compositions that comprise at least two, three, four five, six, seven, eight, nine or ten different Neisseria antigens (one of which is the NspA of the invention; classified as either an adhesin or outer membrane protein) selected from at least one, two, three, four or five different groups of proteins selected from the following:
at least one Neisserial adhesin selected from the group consisting of FhaB, Pi1C, Hsf, Hap, MafA, MafB, Omp26, NMB 0315, NMB 0995, NMB 1119 and NadA;
at least one Neisserial autotransporter selected from the group consisting of Hsf, Hap, IgA protease, AspA, and NadA;
at least one Neisserial toxin selected from the group consisting of FrpA, FrpC, FrpA/C, VapD, NM-ADPRT and either or both of LPS immunotype L2 and LPS immunotype L3;
at least one Neisserial Fe acquisition protein selected from the group consisting of TbpA, TbpB, LbpA, LbpB, HpuA, HpuB, Lipo28 (GNA2132), Sibp, NMB0964, NMB0293, FbpA, Bcp, BfrA, BfrB and P2086 (XthA); and
at least one Neisserial membrane-associated protein, preferably outer membrane protein, particularly integral outer membrane protein, selected from the group consisting of Pi1Q, OMP85, FhaC, TbpA, LbpA, TspA, TspB, TdfH, PorB, M1tA, HpuB, HimD, H isD, GNA1870, OstA, H1pA (GNA1946), NMB 1124, NMB 1162, NMB 1220, NMB 1313, NMB 1953, HtrA, and P1dA (OmplA).
   preferably:
at least one Neisserial adhesin selected from the group consisting of FhaB, Hsf and NadA;
at least one Neisserial autotransporter selected from the group consisting of Hsf, Hap and NadA;
at least one Neisserial toxin selected from the group consisting of FrpA, FrpC, and either or both of LPS immunotype L2 and LPS immunotype L3;
at least one Neisserial Fe acquisition protein selected from the group consisting of TbpA, TbpB, LbpA and LbpB; and
at least one Neisserial outer membrane protein selected from the group consisting of TspA, TspB, Pi1Q, OMP85, and P1dA.

Preferably the first four (and most preferably all five) groups of antigen are represented in the immunogenic composition of the invention.

As previously mentioned where a protein is specifically mentioned herein, it is preferably a reference to a native, full-length protein but it may also encompass antigenic fragments thereof (particularly in the context of subunit vaccines). These are fragments containing or comprising at least 10 amino acids, preferably 20 amino acids, more preferably 30 amino acids, more preferably 40 amino acids or most preferably 50 amino acids, taken contiguously from the amino acid sequence of the protein. In addition, antigenic fragments denotes fragments that are immunologically reactive with antibodies generated against the Neisserial proteins or with antibodies generated by infection of a mammalian host with Neisseria. Antigenic fragments also includes fragments that when administered at an effective dose, elicit a protective immune response against Neisserial infection, more preferably it is protective against N. meningitidis and/or N. gonorrhoeae infection, most preferably it is protective against N. meningitidis serogroup B infection.

Also included in the invention are recombinant fusion proteins of Neisserial proteins of the invention, or fragments thereof. These may combine different Neisserial proteins or fragments thereof in the same protein. Alternatively, the invention also includes individual fusion proteins of Neisserial proteins or fragments thereof, as a fusion protein with heterologous sequences such as a provider of T-cell epitopes, or viral surface proteins such as influenza virus haemagglutinin, tetanus toxoid, diphtheria toxoid, CRM197.

### Addition Antigens of the Invention

NMB references refer to reference numbers to sequences which can be accessed from www.neisseria.org.

### 1. Adhesins

Adhesins include FhaB (WO98/02547), NadA (J. Exp. Med (2002) 195:1445; NMB 1994), Hsf also known as NhhA (NMB 0992) (WO99/31132), Hap (NMB 1985)(WO99/55873), NspA (WO96/29412), MafA (NMB 0652) and MafB (NMB 0643) (Annu Rev Cell Dev Biol. 16; 423-457 (2000); Nature Biotech 20; 914-921 (2002)), Omp26 (NMB 0181), NMB 0315, NMB 0995, NMB 1119 and Pi1C (Mol. Microbio1.1997, 23; 879-892). These are proteins that are involved in the binding of Neisseria to the surface of host cells. Hsf is an example of an adhesin, as well as being an autotransporter protein. Immunogenic compositions of the invention may therefore include combinations of Hsf and other autotransporter proteins where Hsf contributes in its capacity as an adhesin. These adhesins may be derived from Neisseria meningitidis or Neisseria gonorrhoeae or other Neisserial strains. The invention also includes other adhesins from Neisseria.

### FhaB

This antigen has been described in WO98/02547 SEQ ID NO 38 (nucleotides 3083-9025)—see also NMB0497. The present inventors have found FhaB to be particularly effectively at inducing anti-adhesive antibodies alone and in particular with other antigens of the invention. Although full length FhaB could be used, the inventors have found that particular C-terminal truncates are surprisingly at least as effective and preferably even more effective in terms of cross-strain effect. Such truncates have also been advantageously shown to be far easier to clone. FhaB truncates of the invention typically correspond to the N-terminal two-thirds of the FhaB molecule, preferably the new C-terminus being situated at position 1200-1600, more preferably at position 1300-1500, and most preferably at position 1430-1440. Specific embodiments have the C-terminus at 1433 or 1436. Accordingly such FhaB truncates of the invention and vaccines comprising such truncates are preferred components of the combination immunogenic compositions of the invention. The N-terminus may also be truncated by up to 10, 20, 30, 40 or 50 amino acids.

### 2. Autotransporter Proteins

Autotransporter proteins typically are made up of a signal sequence, a passenger domain and an anchoring domain for attachment to the outer membrane. Examples of autotransporter proteins include Hsf (WO99/31132) (NMB 0992), HMW, Hia (van Ulsen et al Immunol Med. Microbiol. 2001 32; 53-64), Hap (NMB 1985) (WO99/55873; van Ulsen et al Immunol Med. Microbiol. 2001 32; 53-64), UspA, UspA2, NadA (NMB 1994) (Comanducci et al J. Exp. Med. 2002 195; 1445-1454), AspA (Infection and Immunity 2002, 70(8); 4447-4461; NMB 1029), Aida-1 like protein, SSh-2 and Tsh. NadA (J. Exp. Med (2002) 195:1445) is another example of an autotransporter proteins, as well as being an adhesin Immunogenic compositions of the invention may therefore include combinations of NadA and adhesins where NadA contributes in its capacity as an autotransporter protein. These proteins may be derived from Neisseria meningitidis or Neisseria gonorrhoeae or other Neiserial strains. The invention also includes other autotransporter proteins from Neisseria.

### Hsf

Hsf has a structure that is common to autotransporter proteins. For example, Hsf from N. meningitidis strain H44/76 consists of a signal sequence made up of amino acids 1-51, a head region at the amino terminus of the mature protein (amino acids 52-479) that is surface exposed and contains variable regions (amino acids 52-106, 121-124, 191-210 and 230-234), a neck region (amino acids 480-509), a hydrophobic alpha-helix region (amino acids 518-529) and an anchoring domain in which four transmembrane strands span the outer membrane (amino acids 539-591).

Although full length Hsf may be used in immunogenic compositions of the invention, various Hsf truncates and deletions may also be advantageously used depending on the type of vaccine.

Where Hsf is used in a subunit vaccine, it is preferred that a portion of the soluble passenger domain is used; for instance the complete domain of amino acids 52 to 479, most preferably a conserved portion thereof, for instance the particularly advantageous sequence of amino acids 134 to 479. Preferred forms of Hsf may be truncated so as to delete variable regions of the protein disclosed in WO01/55182. Preferred variants would include the deletion of one, two, three, four, or five variable regions as defined in WO01/55182. The above sequences and those described below, can be extended or truncated by up to 1, 3, 5, 7, 10 or 15 amino acids at either or both N or C termini.

Preferred fragments of Hsf therefore include the entire head region of Hsf, preferably containing amino acids 52-473. Additional preferred fragments of Hsf include surface exposed regions of the head including one or more of the following amino acid sequences; 52-62, 76-93, 116-134, 147-157, 157-175, 199-211, 230-252, 252-270, 284-306, 328-338, 362-391, 408-418, 430-440 and 469-479.

Where Hsf is present in an outer membrane vesicle preparation, it may be expressed as the full-length protein or preferably as an advantageous variant made up of a fusion of amino acids 1-51 and 134-591 (yielding a mature outer membrane protein of amino acid sequence 134 to the C-terminus). Preferred forms of Hsf may be truncated so as to delete variable regions of the protein disclosed in WO01/55182. Preferred variants would include the deletion of one, two, three, four, or five variable regions as defined in WO01/55182. Preferably the first and second variable regions are deleted. Preferred variants would delete residues from between amino acid sequence 52 through to 237 or 54 through to 237, more preferably deleting residues between amino acid 52 through to 133 or 55 through to 133. The mature protein would lack the signal peptide.

### Hap

Computer analysis of the Hap-like protein from Neisseria meningitidis reveals at least three structural domains. Considering the Hap-like sequence from strain H44/76 as a reference, Domain 1, comprising amino-acid 1 to 42, encodes a sec-dependant signal peptide characteristic of the auto-transporter family, Domain 2, comprising amino-acids 43 to 950, encode the passenger domain likely to be surface exposed and accessible to the immune system, Domain 3, comprising residues 951 to the C-terminus (1457), is predicted to encode a beta-strands likely to assemble into a barrel-like structure and to be anchored into the outer-membrane. Since domains 2 is likely to be surface-exposed, well conserved (more than 80% in all strain tested) and could be produced as subunit antigens in E. coli, it represents an interesting vaccine candidates. Since domains 2 and 3 are likely to be surface-exposed, are well conserved (Pizza et al. (2000), Science 287: 1816-1820), they represent interesting vaccine candidates. Domain 2 is known as the passenger domain.

Immunogenic compositions of the invention may comprise the full-length Hap protein, preferably incorporated into an OMV preparation Immunogenic compositions of the invention may also comprise the passenger domain of Hap which in strain H44/76 is composed of amino acid residues 43-950. This fragment of Hap would be particularly advantageously used in a subunit composition of the invention. The above sequence for the passenger domain of Hap can be extended or truncated by up to 1, 3, 5, 7, 10, 15, 20, 25, or 30 amino acids at either or both N or C termini.

### 3. Iron Acquisition Proteins

Iron aquisition proteins include TbpA (NMB 0461) (WO92/03467, U.S. Pat. No. 5,912,336, WO93/06861 and EP586266), TbpB (NMB 0460) (WO93/06861 and EP586266), LbpA (NMB 1540) (Med Microbiol (1999) 32:1117), LbpB (NMB 1541)(W0/99/09176), HpuA (U73112.2) (Mol. Microbiol. 1997, 23; 737-749), HpuB (NC_{—}003116.1) (Mol. Microbiol. 1997, 23; 737-749), P2086 also known as XthA (NMB 0399) (13^{th} International Pathogenic Neisseria Conference 2002), FbpA (NMB 0634), FbpB, BfrA (NMB 1207), BfrB (NMB 1206), Lipo28 also known as GNA2132 (NMB 2132), Sibp (NMB 1882), HmbR, HemH, Bcp (NMB 0750), Iron (III) ABC transporter-permease protein (Tettelin et al Science 287; 1809-1815 2000), Iron (III) ABC transporter—periplasmic (Tettelin et al Science 287; 1809-1815 2000), TonB-dependent receptor (NMB 0964 and NMB 0293)(Tettelin et al Science 287; 1809-1815 2000) and transferrin binding protein related protein (Tettelin et al Science 287; 1809-1815 2000). These proteins may be derived from Neisseria meningitidis, Neisseria gonorrhoeae or other Neisserial strains. The invention also includes other iron aquisition proteins from Neisseria.

### TbpA

TbpA interacts with TbpB to form a protein complex on the outer membrane of Neisseria, which binds transferrin. Structurally, TbpA contains an intracellular N-terminal domain with a TonB box and plug domain, multiple transmembrane beta strands linked by short intracellular and longer extracellular loops.

Two families of TbpB have been distinguished, having a high molecular weight and a low molecular weight respectively. High and low molecular weight forms of TbpB associate with different families of TbpA which are distinguishable on the basis of homology. Despite being of similar molecular weight, they are known as the high molecular weight and low molecular weight families because of their association with the high or low molecular weight form of TbpB (Rokbi et al FEMS Microbiol. Lett. 100; 51, 1993). The terms TbpA(high) and TbpA(low) are used to refer to these two forms of TbpA, and similarly for TbpB Immunogenic compositions of the invention may comprise TbpA and TbpB from serogroups A, B, C, Y and W-135 of N. meningitidis as well as iron acquisition proteins from other bacteria including N. gonorrhoeae. Transferrin binding proteins TbpA and TbpB have also been referred to as Tbpl and Tbp2 respectively (Cornelissen et al Infection and Immunity 65; 822, 1997).

TbpA contains several distinct regions. For example, in the case of TbpA from N. meningitidis strain H44/76, the amino terminal 186 amino acids form an internal globular domain, 22 beta strands span the membrane, forming a beta barrel structure. These are linked by short intracellular loops and larger extracellular loops. Extracellular loops 2, 3 and 5 have the highest degree of sequence variability and loop 5 is surface exposed. Loops 5 and 4 are involved in ligand binding.

Preferred fragments of TbpA include the extracellular loops of TbpA. Using the sequence of TbpA from N. meningitidis strain H44/76, these loops correspond to amino acids 200-202 for loopl, amino acids 226-303 for loop 2, amino acids 348-395 for loop 3, amino acids 438-471 for loop 4, amino acids 512-576 for loop 5, amino acids 609-625 for loop 6, amino acids 661-671 for loop 7, amino acids 707-723 for loop 8, amino acids 769-790 for loop 9, amino acids 814-844 for loop 10 and amino acids 872-903 for loop 11. The corresponding sequences, after sequence alignment, in other Tbp proteins would also constitute preferred fragments. Most preferred fragments would include amino acid sequences constituting loop 2, loop 3, loop 4 or loop 5 of Tbp.

Where the immunogenic compositions of the invention comprise TbpA, it is preferable to include both TbpA(high) and TbpA (low).

Although TbpA is preferably presented in an OMV vaccine, it may also be part of a subunit vaccine. For instance, isolated iron acquisition proteins which could be introduced into an immunogenic composition of the invention are well known in the art (WO00/25811). They may be expressed in a bacterial host, extracted using detergent (for instance 2% Elugent) and purified by affinity chromatography or using standard column chromatography techniques well known to the art (Oakhill et al Biochem J. 2002 364; 613-6).

Where TbpA is presented in an OMV vaccine, its expression can be upregulated by genetic techniques discussed herein or in WO 01/09350, or may preferably be upregulated by growth of the parent strain under iron limitation conditions. This process will also result in the upregulation of variable iron-regulated proteins, particularly FrpB which may become immunodominant and it is therefore advantageous to downregulate the expression of (and preferably delete the genes encoding) such proteins (particularly FrpB) as described in WO 01/09350, to ensure that the immunogenic composition of the invention elicits an immune response against antigens present in a wide range of Neisserial strains. It is preferred to have both TbpA(high) and TbpA(low) present in the immunogenic composition and this is preferably achieved by combining OMVs derived from two strains, expressing the alternative forms of TbpA.

### 4. Toxins

Toxins include FrpA (NMB 0585; NMB 1405), FrpA/C (see below for definition), FrpC (NMB 1415; NMB 1405) (WO92/01460), NM-ADPRT (NMB 1343) (13^{th} International Pathogenic Neisseria Conference 2002 Masignani et al p135), VapD (NMB 1753), lipopolysaccharide (LPS; also called lipooligosaccharide or LOS) immunotype L2 and LPS immunotype L3. FrpA and FrpC contain a region which is conserved between these two proteins and a preferred fragment of the proteins would be a polypeptide containing this conserved fragment, preferably comprising amino acids 227-1004 of the sequence of FrpA/C. These antigens may be derived from Neisseria meningitidis or Neisseria gonorrhoeae or other Neisserial strains. The invention also includes other toxins from Neisseria.

In an alternative embodiment, toxins may include antigens involved in the regulation of toxicity, for example OstA which functions in the synthesis of lipopolysaccharides.

### FrpA and FrpC

Neisseria meningitidis encodes two RTX proteins, referred to as FrpA & FrpC secreted upon iron limitation (Thompson et al., (1993) J. Bacteriol. 175:811-818; Thompson et al., (1993) Infect. Immun. 61:2906-2911). The RTX (Repeat ToXin) protein family have in common a series of 9 amino acid repeat near their C-termini with the consensus: Leu Xaa Gly Gly Xaa Gly (Asn/Asp) Asp Xaa. (LXGGXGN_{/D}DX; SEQ ID NO:1). The repeats in E. coli H1yA are thought to be the site of Ca2+binding. As represented in FIG. 4, meningococcal FrpA and FrpC proteins, as characterized in strain FAM20, share extensive amino-acid similarity in their central and C-terminal regions but very limited similarity (if any) at the N-terminus Moreover, the region conserved between FrpA and FrpC exhibit some polymorphism due to repetition (13 times in FrpA and 43 times in FrpC) of a 9 amino acid motif.

Immunogenic compositions of the invention may comprise the full length FrpA and/or FrpC or preferably, a fragment comprising the sequence conserved between FrpA and FrpC. The conserved sequence is made up of repeat units of 9 amino acids. Immunogenic compositions of the invention would preferably comprise more that three repeats, more than 10 repeats, more than 13 repeats, more than 20 repeats or more than 23 repeats.

Such truncates have advantageous properties over the full length molecules, and vaccines comprising such antigens are preferred for being incorporated in the immunogenic compositions of the invention.

Sequences conserved between FrpA and FrpC are designated FrpA/C and wherever FrpA or FrpC forms a constituent of immunogenic compositions of the invention, FrpA/C could be advantageously used. Amino acids 277-1004 of the FrpA sequence is the preferred conserved region. The above sequence can be extended or truncated by up to 1, 3, 5, 7, 10, 15, 20, 25, or 30 amino acids at either or both N or C termini.

### LPS

LPS (lipopolysaccharide, also known as LOS-lipooligosaccharide) is the endotoxin on the outer membrane of Neisseria. The polysaccharide moiety of the LPS is known to induce bactericidal antibodies.

Heterogeneity within the oligosaccharide moiety of the LPS generates structural and antigenic diversity among different neisserial strains (Griffiss et al. Inf. Immun. 1987; 55: 1792-1800). This has been used to subdivide meningococcal strains into 12 immunotypes (Scholtan et al. J Med Microbiol 1994, 41:236-243) Immunotypes L3, L7, & L9 are immunologically identical and are structurally similar (or even the same) and have therefore been designated L3,7,9 (or, for the purposes of this specification, generically as “L3”). Meningococcal LPS L3,7,9 (L3), L2 and L5 can be modified by sialylation, or by the addition of cytidine 5′-monophosphate-N-acetylneuraminic acid. Although L2, L4 and L6 LPS are distinguishable immunologically, they are structurally similar and where L2 is mentioned herein, either L4 or L6 may be optionally substituted within the scope of the invention. See M. P. Jennings et al, Microbiology 1999, 145, 3013-3021 and Mol Microbiol 2002, 43:931-43 for further illustration of LPS structure and heterogeneity.

Where LPS, preferably meningococcal LPS, is included in a vaccine of the invention, preferably and advantageously either or both of immunotypes L2 and L3 are present. LPS is preferably presented in an outer membrane vesicle (preferably where the vesicle is extracted with a low percentage detergent, more preferably 0-0.5%, 0.02-0.4%, 0.04-0.3%, 0.06-0.2%, 0.08-0.15% or 0.1%, most preferably deoxycholate [DOC]) but may also be part of a subunit vaccine. LPS may be isolated using well known procedure including the hot water-phenol procedure (Wesphal and Jann Meth. Carbo. Chem. 5; 83-91 1965). See also Galanos et al. 1969, Eur J Biochem 9:245-249, and Wu et al. 1987, Anal Bio Chem 160:281-289. LPS may be used plain or conjugated to a source of T-cell epitopes such as tetanus toxoid, Diphtheria toxoid, CRM-197 or OMV outer membrane proteins. Techniques for conjugating isolated LOS are also known (see for instance EP 941738 incorporated by reference herein).

Where LOS (in particular the LOS of the invention) is present in a bleb formulation the LOS is preferably conjugated in situ by methods allowing the conjugation of LOS to one or more outer membrane proteins also present on the bleb preparation (e.g. PorA or PorB in meningococcus).

This process can advantageously enhance the stability and/or immunogenicity (providing T-cell help) and/or antigenicity of the LOS antigen within the bleb formulation—thus giving T-cell help for the T-independent oligosaccharide immunogen in its most protective conformation—as LOS in its natural environment on the surface of meningococcal outer membrane. In addition, conjugation of the LOS within the bleb can result in a detoxification of the LOS (the Lipid A portion being stably buried in the outer membrane thus being less available to cause toxicity). Thus the detoxification methods mentioned herein of isolating blebs from htrB⁻ or msbB⁻ mutants, or by adding non toxic peptide functional equivalent of polymyxin B [a molecule with high affinity to Lipid A] to the composition (see WO 93/14115, WO 95/03327, Velucchi et al (1997) J Endotoxin Res 4: 1-12, and EP 976402 for further details of non-toxic peptide functional equivalents of polymyxin B—particularly the use of the peptide SAEP 2 (of sequence KTKCKFLKKC-SEQ ID NO: 2) where the 2 cysteines form a disulphide bridge)) may not be required (but which may be added in combination for additional security). Thus the inventors have found that a composition comprising blebs wherein LOS present in the blebs has been conjugated in an intra-bleb fashion to outer membrane proteins also present in the bleb can form the basis of a vaccine for the treatment or prevention of diseases caused by the organism from which the blebs have been derived, wherein such vaccine is substantially non-toxic and is capable of inducing a T-dependent bactericidal response against LOS in its native environment.

Such bleb preparations may be isolated from the bacterial in question (see WO 01/09350), and then subjected to known conjugation chemistries to link groups (e.g. NH₂ or COOH) on the oligosaccharide portion of LOS to groups (e.g. NH₂ or COOH) on bleb outer membrane proteins. Cross-linking techniques using glutaraldehyde, formaldehyde, or glutaraldehyde/formaldehyde mixes may be used, but it is preferred that more selective chemistries are used such as EDAC or EDAC/NHS (J. V. Staros, R. W. Wright and D. M. Swingle. Enhancement by N-hydroxysuccinimide of water-soluble carbodiimide-mediated coupling reactions. Analytical Chemistry 156: 220-222 (1986); and Bioconjugates Techniques. Greg T. Hermanson (1996) pp 173-1′76). Other conjugation chemistries or treatments capable of creating covalent links between LOS and protein molecules that could be used are described in EP 941738.

Preferably the bleb preparations are conjugated in the absence of capsular polysaccharide. The blebs may be isolated from a strain which does not produce capsular polysaccharide (naturally or via mutation as described below), or may be purified from most and preferably all contaminating capsular polysaccharide. In this way, the intra-bleb LOS conjugation reaction is much more efficient.

Preferably more than 10, 20, 30, 40, 50, 60, 70, 80, 90, or 95% of the LOS present in the blebs is cross-linked/conjugated.

Intrableb conjugation should preferably incorporate 1, 2 or all 3 of the following process steps: conjugation pH should be greater than pH 7.0, preferably greater than or equal to pH 7.5 (most preferably under pH 9); conditions of 1-5% preferably 2-4% most preferably around 3% sucrose should be maintained during the reaction; NaCl should be minimised in the conjugation reaction, preferably under 0.1M, 0.05M, 0.01M, 0.005M, 0.001M, and most preferably not present at all. All these process features make sure that the blebs remain stable and in solution throughout the conjugation process.

The EDAC/NHS conjugation process is a preferred process for intra-bleb conjugation. EDAC/NHS is preferred to formalydehyde which can cross-link to too high an extent thus adversely affecting filterability. EDAC reacts with carboxylic acids (such as KDO in LOS) to create an active-ester intermediate. In the presence of an amine nucleophile (such as lysines in outer membrane proteins such as PorB), an amide bond is formed with release of an isourea by-product. However, the efficiency of an EDAC-mediated reaction may be increased through the formation of a Sulfo-NHS ester intermediate. The Sulfo-NHS ester survives in aqueous solution longer than the active ester formed from the reaction of EDAC alone with a carboxylate. Thus, higher yields of amide bond formation may be realized using this two-stage process. EDAC/NHS conjugation is discussed in J. V. Staros, R. W. Wright and D. M. Swingle. Enhancement by N-hydroxysuccinimide of water-soluble carbodiimide-mediated coupling reactions. Analytical chemistry 156: 220-222 (1986); and Bioconjugates Techniques. Greg T. Hermanson (1996) pp 173-176. Preferably 0.01-5 mg EDAC/mg bleb is used in the reaction, more preferably 0.05-1 mg EDAC/mg bleb. The amount of EDAC used depends on the amount of LOS present in the sample which in turn depends on the deoxycholate (DOC) % used to extract the blebs. At low % DOC (e.g. 0.1%), high amounts of EDAC are used (1 mg/mg and beyond), however at higher % DOC (e.g. 0.5%), lower amounts of EDAC are used (0.025-0.1 mg/mg) to avoid too much inter-bleb crosslinking.

A preferred process of the invention is therefore a process for producing intra-bleb conjugated LOS (preferably meningococcal) comprising the steps of conjugating blebs in the presence of EDAC/NHS at a pH between pH 7.0 and pH 9.0 (preferably around pH 7.5), in 1-5% (preferably around 3%) sucrose, and optionally in conditions substantially devoid of NaCl (as described above), and isolating the conjugated blebs from the reaction mix.

The reaction may be followed on Western separation gels of the reaction mixture using anti-LOS (e.g. anti-L2 or anti-L3) mAbs to show the increase of LOS molecular weight for a greater proportion of the LOS in the blebs as reaction time goes on.

Yields of 99% blebs can be recovered using such techniques.

EDAC was found to be an excellent intra-bleb cross-linking agent in that it cross-linked LOS to OMP sufficiently for improved LOS T-dependent immunogenicity, but did not cross link it to such a high degree that problems such as poor filterability, aggregation and inter-bleb cross-linking occurred. The morphology of the blebs generated is similar to that of unconjugated blebs (by electron microscope). In addition, the above protocol avoided an overly high cross-linking to take place (which can decrease the immunogenicity of protective OMPs naturally present on the surface of the bleb e.g. TbpA or Hsf).

It is preferred that the meningococcal strain from which the blebs are derived is a mutant strain that cannot produce capsular polysaccharide (in particular siaD⁻). It is also preferred that immunogenic compositions effective against meningococcal disease comprise both an L2 and an L3 bleb, wherein the L2 and L3 LOS are both conjugated to bleb outer membrane proteins. Furthermore, it is preferred that the LOS structure within the intra-bleb conjugated bleb is consistent with it having been derived from an lgtE⁻or, preferably, lgtB⁻ meningococcal strain. Most preferably immunogenic compositions comprise intrableb-conjugated blebs: derived from a mutant meningococcal strain that cannot produce capsular polysaccharide and is lgtB⁻; comprising L2 and L3 blebs derived from mutant meningococcal strains that cannot produce capsular polysaccharide; comprising L2 and L3 blebs derived from mutant meningococcal strains that are lgtB⁻; or most preferably comprising L2 and L3 blebs derived from mutant meningococcal strains that cannot produce capsular polysaccharide and are lgtB⁻.

Typical L3 meningococcal strain that can be used for the present invention is H44/76 menB strain. A typical L2 strain is the B16B6 menB strain or the 39E meningococcus type C strain.

As stated above, the blebs of the invention have been detoxified to a degree by the act of conjugation, and need not be detoxified any further, however further detoxification methods may be used for additional security, for instance using blebs derived from a meningococcal strain that is htrB⁻ or msbB⁻ or adding a non-toxic peptide functional equivalent of polymyxin B [a molecule with high affinity to Lipid A] (preferably SEAP 2) to the bleb composition (as described above).

In the above way meningococcal blebs and immunogenic compositions comprising blebs are provided which have as an important antigen LOS which is substantially non-toxic, devoid of autoimmunity problems, has a T-dependent character, is present in its natural environment, and is capable of inducing a bactericidal antibody response against more than 90% of meningococcal strains (in the case of L2+L3 compositions).

### 5. Integral Outer Membrane Proteins

Other categories of Neisserial proteins may also be candidates for inclusion in the Neisserial vaccines of the invention and may be able to combine with other antigens in a surprisingly effective manner. Membrane associated proteins, particularly integral membrane proteins and most advantageously outer membrane proteins, especially integral outer membrane proteins may be used in the compositions of the present invention. An example of such a protein is P1dA also known as OmplA (NMB 0464) (WO00/15801) which is a Neisserial phospholipase outer membrane protein. Further examples are TspA (NMB 0341) (Infect. Immun. 1999, 67; 3533-3541) and TspB (T-cell stimulating protein) (WO 00/03003; NMB 1548, NMB 1628 or NMB 1747). Further examples include Pi1Q (NMB 1812) (WO99/61620), OMP85—also known as D15-(NMB 0182) (WO00/23593), NspA (U52066) (WO96/29412), FhaC(NMB 0496 or NMB 1780), PorB (NMB 2039) (Mol. Biol. Evol. 12; 363-370, 1995), HpuB (NC_{—}003116.1), TdfH(NMB 1497) (Microbiology 2001, 147; 1277-1290), OstA (NMB 0280), M1tA also known as GNA33 and Lipo30 (NMB0033), HtrA (NMB 0532; WO 99/55872), HimD (NMB 1302), H isD (NMB 1581), GNA 1870 (NMB 1870), H1pA (NMB 1946), NMB 1124, NMB 1162, NMB 1220, NMB 1313, NMB 1953, HtrA, TbpA (NMB 0461) (WO92/03467) (see also above under iron acquisition proteins) and LbpA (NMB 1541).

### OMP85

OMP85/D15 is an outer membrane protein having a signal sequence, a N-terminal surface-exposed domain and an integral membrane domain for attachment to the outer membrane Immunogenic compositions of the invention may also comprise the full length OMP85, preferably as part of an OMV preparation. Fragments of OMP85 may also be used in immunogenic compositions of the invention, in particularly, the N terminal surface-exposed domain of OMP85 made up of amino acid residues 1-475 or 50-475 is preferably incorporated into a subunit component of the immunogenic compositions of the invention. The above sequence for the N terminal surface-exposed domain of OMP85 can be extended or truncated by up to 1, 3, 5, 7, 10, 15, 20, 25, or 30 amino acids at either or both N or C termini. It is preferred that the signal sequence is omitted from the OMP85 fragment.

### OstA

OstA functions in the synthesis of lipopolysaccharides and may be considered to be a regulator of toxicity. OstA may alternatively be included in the toxin category where the toxin category is broadened to contain regulators of toxicity as well as toxins.

The immunogenic compositions of the invention may comprise antigens (proteins, LPS and polysaccharides) derived from Neisseria meningitidis serogroups A, B, C, Y, W-135 or Neisseria gonorrhoeae.

Preferably the subunit composition comprises NspA of the present invention and at least one further antigen selected from the following list: FhaB, passenger domain of Hsf, passenger domain of Hap, N-terminal surface exposed domain of OMP85, FrpA, FrpC, FrpA/C, TbpB, LbpB, P1dA, Pi1Q and either or both of LPS immunotype L2 and LPS immunotype L3.

Preferably the subunit composition (comprising refolded NspA, or subunit combinations described above) further comprises a Neisserial (preferably meningococcal) outer membrane vesicle (OMV) preparation. Preferably the OMV preparation has at least one antigen (more preferably 2, 3, 4 or 5) selected from the following list which has been recombinantly upregulated in the outer membrane vesicle: NspA, Hsf, Hap, OMP85, TbpA (high), TbpA (low), LbpA, TbpB, LbpB, Pi1Q and P1dA; and optionally comprising either or both of LPS immunotype L2 and LPS immunotype L3.

Preferably the composition comprises a subunit composition comprising NspA and an outer membrane vesicle preparation wherein the subunit composition further comprises at least one antigen selected from the following list: FhaB, passenger domain of Hsf, passenger domain of Hap, N-terminal surface exposed domain of OMP85, FrpA, FrpC, FrpA/C, TbpB, LbpB, Pi1Q and the outer membrane vesicle preparation having at least one different antigen selected from the following list, which has been recombinantly upregulated in the outer membrane vesicle: NspA, Hsf, Hap, OMP85, TbpA (high), TbpA (low), LbpA, TbpB, LbpB, Pi1Q and P1dA; and optionally comprising either or both of LPS immunotype L2 and LPS immunotype L3.

### Further Combinations

The immunogenic composition of the invention may further comprise bacterial capsular polysaccharides or oligosaccharides. The capsular polysaccharides or oligosaccharides may be derived from one or more of: Neisseria meningitidis serogroup A, C, Y, and/or W-135, Haemophilus influenzae b, Streptococcus pneumoniae, Group A Streptococci, Group B Streptococci, Staphylococcus aureus and Staphylococcus epidermidis.

A further aspect of the invention are vaccine combinations comprising the antigenic composition of the invention with other antigens which are advantageously used against certain disease states including those associated with viral or Gram positive bacteria.

In one preferred combination, the antigenic compositions of the invention are formulated with 1, 2, 3 or preferably all 4 of the following meningococcal capsular polysaccharides or oligosaccharides which may be plain or conjugated to a protein carrier: A, C, Y or W-135. Preferably the immunogenic compositions of the invention are formulated with A and C; or C; or C and Y. Such a vaccine containing proteins from N. meningitidis serogroup B may be advantageously used as a global meningococcus vaccine.

In a further preferred embodiment, the antigenic compositions of the invention, preferably formulated with 1, 2, 3 or all 4 of the plain or conjugated meningococcal capsular polysaccharides or oligosaccharides A, C, Y or W-135 (as described above), are formulated with a conjugated H. influenzae b capsular polysaccharide (or oligosaccharides), and/or one or more plain or conjugated pneumococcal capsular polysaccharides (or oligosaccharides) (for instance those described below). Optionally, the vaccine may also comprise one or more protein antigens that can protect a host against Streptococcus pneumoniae infection. Such a vaccine may be advantageously used as a global meningitis vaccine.

In a still further preferred embodiment, the immunogenic composition of the invention is formulated with capsular polysaccharides or oligosaccharides derived from one or more of Neisseria meningitidis, Haemophilus influenzae b, Streptococcus pneumoniae, Group A Streptococci, Group B Streptococci, Staphylococcus aureus or Staphylococcus epidermidis. The pneumococcal capsular polysaccharide or oligosaccharides antigens are preferably selected from serotypes 1, 2, 3, 4, 5, 6B, 7F, 8, 9N, 9V, 10A, 11A, 12F, 14, 15B, 17F, 18C, 19A, 19F, 20, 22F, 23F and 33F (most preferably from serotypes 1, 3, 4, 5, 6B, 7F, 9V, 14, 18C, 19F and 23F). A further preferred embodiment would contain the PRP capsular polysaccharide or oligosaccharide of Haemophilus influenzae. A further preferred embodiment would contain the Type 5, Type 8 or 336 capsular polysaccharides of Staphylococcus aureus. A further preferred embodiment would contain the Type I, Type II or Type III capsular polysaccharides of Staphylococcus epidermidis. A further preferred embodiment would contain the Type Ia, Type Ic, Type II or Type III capsular polysaccharides of Group B streptococcus. A further preferred embodiment would contain the capsular polysaccharides of Group A streptococcus, preferably further comprising at least one M protein and more preferably multiple types of M protein.

Such capsular polysaccharides or oligosaccharides of the invention may be unconjugated or conjugated to a carrier protein such as tetanus toxoid, tetanus toxoid fragment C, diphtheria toxoid, CRM197, pneumolysis, Protein D (U.S. Pat. No. 6,342,224). The polysaccharide or oligosaccharide conjugate may be prepared by any known coupling technique. For example the polysaccharide can be coupled via a thioether linkage. This conjugation method relies on activation of the polysaccharide with 1-cyano-4-dimethylamino pyridinium tetrafluoroborate (CDAP) to form a cyanate ester. The activated polysaccharide may thus be coupled directly or via a spacer group to an amino group on the carrier protein. Preferably, the cyanate ester is coupled with hexane diamine and the amino-derivatised polysaccharide is conjugated to the carrier protein using heteroligation chemistry involving the formation of the thioether linkage. Such conjugates are described in PCT published application WO93/15760 Uniformed Services University.

The conjugates can also be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-O-477508. A further method involves the coupling of a cyanogen bromide activated polysaccharide derivatised with adipic acid hydrazide (ADH) to the protein carrier by Carbodiimide condensation (Chu C. et al Infect. Immunity, 1983 245 256).

Preferred pneumococcal proteins antigens are those pneumococcal proteins which are exposed on the outer surface of the pneumococcus (capable of being recognised by a host's immune system during at least part of the life cycle of the pneumococcus), or are proteins which are secreted or released by the pneumococcus. Most preferably, the protein is a toxin, adhesin, 2-component signal tranducer, or lipoprotein of Streptococcus pneumoniae, or fragments thereof. Particularly preferred proteins include, but are not limited to: pneumolysin (preferably detoxified by chemical treatment or mutation) [Mitchell et al. Nucleic Acids Res. 1990 Jul. 11; 18(13): 4010 “Comparison of pneumolysin genes and proteins from Streptococcus pneumoniae types 1 and 2.”, Mitchell et al. Biochim Biophys Acta 1989 Jan. 23; 1007(1): 67-72 “Expression of the pneumolysin gene in Escherichia coli: rapid purification and biological properties.”, WO 96/05859 (A. Cyanamid), WO 90/06951 (Paton et al), WO 99/03884 (NAVA)]; PspA and transmembrane deletion variants thereof (U.S. Pat. No. 5,804,193-Briles et al.); PspC and transmembrane deletion variants thereof (WO 97/09994-Briles et al); PsaA and transmembrane deletion variants thereof (Berry & Paton, Infect Immun 1996 December; 64(12):5255-62 “Sequence heterogeneity of PsaA, a 37-kilodalton putative adhesin essential for virulence of Streptococcus pneumoniae”); pneumococcal choline binding proteins and transmembrane deletion variants thereof; CbpA and transmembrane deletion variants thereof (WO 97/41151; WO 99/51266); Glyceraldehyde-3-phosphate—dehydrogenase (Infect. Immun. 1996 64:3544); HSP70 (WO 96/40928); PcpA (Sanchez-Beato et al. FEMS Microbiol Lett 1998, 164:207-14); M like protein, (EP 0837130) and adhesin 18627, (EP 0834568). Further preferred pneumococcal protein antigens are those disclosed in WO 98/18931, particularly those selected in WO 98/18930 and PCT/US99/30390.

The immunogenic composition/vaccine of the invention may also optionally comprise outer membrane vesicle preparations made from other Gram negative bacteria, for example Moraxella catarrhalis or Haemophilus influenzae.

### Compositions, Kits and Administration

As previously mentioned the invention provides compositions comprising a NspA protein for administration to a cell or to a multicellular organism.

An immunogenic composition is a composition comprising at least one antigen which is capable of generating an immune response when administered to a host. Preferably, such immunogenic preparations are capable of generating a protective immune response against Neisserial, preferably Neisseria meningitidis and/or Neisseria gonorrhoeae infection.

The invention also relates to compositions comprising a protein discussed herein or their agonists or antagonists. The proteins of the invention may be employed in combination with a non-sterile or sterile carrier or carriers for use with cells, tissues or organisms, such as a pharmaceutical carrier suitable for administration to an individual. Such compositions comprise, for instance, a media additive or a therapeutically effective amount of a protein of the invention and a pharmaceutically acceptable carrier or excipient. Such carriers may include, but are not limited to, saline, buffered saline, dextrose, water, glycerol, ethanol and combinations thereof. The formulation should suit the mode of administration. The invention further relates to diagnostic and pharmaceutical packs and kits comprising one or more containers filled with one or more of the ingredients of the aforementioned compositions of the invention.

Proteins and other compounds of the invention may be employed alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a protein or other compounds of the present invention can be formulated in an enteric or an encapsulated formulation, oral administration may also be possible. Administration of these compounds may also be topical and/or localized, in the form of salves, pastes, gels, solutions, powders and the like.

For administration to mammals, and particularly humans, it is expected that the daily dosage level of the active agent will be from 0.01 mg/kg to 10 mg/kg, typically around 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

The dosage range required depends on the choice of peptide, the route of administration, the nature of the formulation, the nature of the subject's condition, and the judgement of the attending practitioner. Suitable dosages, however, are in the range of 0.1-100 μg/kg of subject.

A vaccine composition is conveniently in injectable form. Conventional adjuvants may be employed to enhance the immune response. A suitable unit dose for vaccination is 0.5-5 microgram/kg of antigen, and such dose is preferably administered 1-3 times and with an interval of 1-3 weeks. With the indicated dose range, no adverse toxicological effects will be observed with the compounds of the invention which would preclude their administration to suitable individuals.

Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Preferred features and embodiment of the present invention will now be described further with reference to the following Examples:

### Example 1

### Preparation of Native Nspa (Neisserial Surface Protein a)

Genomic DNA of Neisseria meningitidis strain H44/76 was prepared using the Quiagen genomic preparation kit. The part of the NspA gene encoding the mature NspA protein, i.e. without the 19 amino acid signal peptide, was PCR amplified from genomic H44/76 DNA using the primer pair 5′ gctacatatggaaggcgcatccggcttttacg (SEQ ID NO:3) and 5′ gctaggatcctcagaatttgacgcgcacaccgg (SEQ ID NO:4).

The resultant PCR product was cloned into pCRII-TOPO (Invitrogen), digested with NdeI and BamHI and ligated into pET11a (Novagen). The resultant plasmid pET11a-NspA was transformed into BL21-DE3 cells (Novagen). Five liters of these cells were grown at 37° C. in Luria Broth containing 100 ug/ml ampicillin. After reaching an OD600 of 0.6, 1 mM of isopropyl-thio-β-D-galactopyranoside (IPTG) was added for 2 hours (alternatively 4 additional hours with 0.1 mM IPTG was used). Cells were harvested and washed with 600 ml of 0.9% (w/v) NaCl. NspA was present as inclusion bodies. The cell pellet was resuspended in 100 ml ice-cold TE buffer (50 mM Tris/HC1+40 mM EDTA pH 8.0). Twenty-five grams of sucrose and 20 mg lysozyme was added for 30 minutes with shaking. A hundred mls of TE was added and shaking was continued for another 30 minutes. Then 100 ml fractions were sonicated (Branson sonifier), 4 mls of 10% BRIJ™ were added with another round of sonication. Suspensions were spun 30 minutes at 4000 rpm. The pellet containing inclusion bodies was washed once with TE buffer, resuspended in 40 mls of 10 mM Tris/HC1 pH 8.3, spun in aliquots (8000 rpm Eppendorf centrifuge) and frozen at −20 C.

Inclusion bodies were solubilized in 20 mM Tris/HC1+0.1 M glycine+8 M urea pH 8.0.

Solubilized, denatured NspA protein was refolded by dilution (1:20) into 20 mM ethanolamine containing 0.5% purified SB-12 (3-dimethyldodecylammoniopropanesulfonate, Fluka—DodMe₂NprSO₃). SB-12 was purified by passing a concentrated solution of the detergent in methanol/chloroform (1:1) over an Al₂O₃ column. Alternatively, a 5-fold dilution into 20 mM ethanolamine, pH 11, containing 1% (w/v) purified SB-12 (3-dimethyldodecylammoniopropanesulfonate, Fluka) was carried out. The solution was left stirring overnight at room temperature.

Refolding was evaluated by semi-native SDS-PAGE (i.e. running with as little as necessary SDS, low amperage, at 4 C), which reveals refolded NspA as a slower running form compared to denatured NspA (5 min boiling with 1% SDS). This is consistent with its running behaviour when present in native cell envelopes as reported in the literature. Judged by silver staining of the gels, 100% refolding efficiency was achieved. Gels usually contained 14% acrylamide, with no SDS in the gel and only 0.03% SDS in the 3× sample buffer (additionally comprising 0.1M Tris pH 6.8, 15.4% glycerol, 7.7%-mercaptoethanol, and 0.008% (w/v) bromophenol blue), and were not heated before electrophoresis.

To further purify the folded NspA, the mixture was loaded onto a 1 ml mono S column (Pharmacia), pre-equilibrated with 10 mM DodMe2NprSO3, 20 mM Tris-HC1 pH 8.5 (buffer A). Prior to loading, the pH of the protein sample was adjusted to pH 8.5 using 1 M HC1. The column was washed with buffer A, and proteins were eluted with a linear gradient of 0-500 mM NaCl in buffer A, total volume 50 ml.

Although OMPs are generally heat modifiable, i.e. folded monomers run faster in SDS-PAGE gels than heat-denatured proteins, in contrast we observed that NspA migrated slower in the gel after refolding. The denatured protein migrated at a mass of ˜18 kDa, whereas the folded protein migrated at 22 kDa, which agrees well with the electrophoretic mobility of native NspA isolated from neisserial membranes (Moe et al. 1999 I&I 67:5664). Analytical ultracentrifugation and chemical cross-linking experiments confirmed that refolded NspA was a monomer (i.e. did not dimerize) in a detergent-containing solution, as confirmed by its crystal structure (see below).

### Example 2

### Evidence of the Properly Folded Structure of Refolded NspA

In addition to the above evidence that refolded NspA migrated on a gel in the same way as native NspA isolated from neisserial membranes, 2 further indicators of proper refolding were carried out.

Refolded NspA was injected into mice, and the resulting sera obtained was able to recognise native (non-denatured) NspA in an ELISA test.

Refolded NspA was also used to generate crystals of high quality from which the crystal structure of folded Neisserial NspA was solved (Vandeputte-Rutten et al. 2003 JBC 278:24825).

### Example 3

### Serum Bactericidal Activity of Antibodies Against NspA in Combination With TbpA and Hsf

N. meningitidis strain H66/76 in which the expression of PorA and the synthesis of capsular polysaccharides were switched off (see WO 01/09350) was used as the background strain for up-regulating the expression of TbpA and Hsf, or of NspA using the procedures described in WO 01/09350. Outer membrane vesicles were prepared from the strains. TbpA may be upregulated genetically or by growing the production strain in low iron conditions.

The outer membrane vesicle preparations were adsorbed onto Al(OH)₃ and injected into mice on days 0, 21 and 28. On day 42, the mice were bled and sera prepared. The sera against TbpA and Hsf up-regulated OMVs were mixed with sera from mice vaccinated with OMVs containing up-regulated NspA and serum bactericidal assays were performed as follows.

The serum bactericidal activity of antisera from the mice inoculated with OMVs were compared in assays using either the homologous strain H44/76 or the heterologous strain Cu385. The serum bactericidal assay has been shown to show good correlation with protection and is therefore a good indication of how effective a candidate composition will be in eliciting a protective immune response.

Neisseria meningitidis serogroup B wild type strains (H44/76 strain=B:15 P1.7,16 L3,7,9 and CU385 strain=B: 4 P1.19,15 L3,7,9) were cultured overnight on MH+1% Polyvitex+1% horse serum Petri dishes at 37° C.+5% CO₂. They were sub-cultured for 3 hours in a liquid TSB medium supplemented with 50 μM of Desferal (Iron chelator) at 37° C. under shaking to reach an optical density of approximately 0.5 at 470 nm.

Pooled or individual sera were inactivated for 40 min at 56° C. Serum samples were diluted 1/100 in HBSS-BSA 0.3% and then serially diluted two fold (8 dilutions) in a volume of 50 μl in round bottom microplates.

Bacteria, at the appropriate OD, were diluted in HBSS-BSA 0.3% to yield 1.3 10e4 CFU per ml. 37.5 μl of this dilution was added to the serum dilutions and microplates were incubated for 15 minutes at 37° C. under shaking. Then, 12.5 μl of rabbit complement were added to each well. After 1 hour of incubation at 37° C. and under shaking, the microplates were placed on ice to stop the killing.

Using the tilt method, 20 μl of each well were plated on MH+1% Polyvitex+1% horse serum Petri dishes and incubated overnight at 37° C.+CO2. The CFU's were counted and the percent of killing calculated. The serum bactericidal titer is the last dilution of serum yielding≧50% killing.

Mice are thought to produce a significant level of bactericidal antibodies if titres are greater than 1/100, particularly in experiments using the heterologous strain.

### Results

Results are shown in the table below. In assays using the homologous H44/76 stain, the addition of antibodies against NspA, did not produce a serum bactericidal titre higher than that produced using antibodies against TbpA and Hsf alone.

However, the addition of antibodies against NspA was advantageous in serum bactericidal assays using a heterologous strain. Antibodies against NspA were effective at increasing the serum bactericidal titre against the CU385 strain.

Serum
Bactericidal Titre

| Antisera Mix | H44/76 | CU385 |
|---|---|---|
| antiTbpA-Hsf and nonimmune sera | 5378 | 2141 |
| anti-TbpA-Hsf and anti-NspA | 4738 | 2518 |

NspA when combined with TbpA and/or Hsf in an effective amount (upregulated) is able to improve the cross-bactericidal character of sera generated against the combined vaccine.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1. A method of preparing a medicament containing an isolated, refolded NspA protein, said method comprising the steps of:
a. optionally expressing an NspA protein in a host cell;
b. optionally breaking the host cell to obtain an inclusion body comprising the NspA protein;
c. optionally washing the inclusion body;
d. optionally solubilizing at least part of the inclusion body and the NspA protein;
e. contacting a solubilized NspA protein with a refolding buffer; and
f. optionally removing the refolding buffer from the NspA protein.

2. The method according to claim 1, wherein said refolding buffer comprises 3-dimethyldodecylammoniopropanesulfonate (SB-12).

3. The method according to claim 2, wherein said refolding buffer additionally comprises ethanolamine.

4. The method according to claim 3 wherein the ethanolamine is present at a concentration of about 20 mM ethanolamine.

5. The method according to claim 1 wherein the refolding buffer has pH 11.

6. The method according to claim 1 wherein the SB-12 is 0.2% SB-12.

7. The method according to claim 1 wherein the SB-12 is 0.5% SB-12.

8. The method according to claim 1 wherein the SB-12 is purified.

9. The method according to claim 8 wherein the SB-12 is purified by passing it over an Al₂O₃ column.

10. A medicament prepared according to the method of claim 1.

11. A medicament according to claim 10 wherein at least 50% of the NspA protein present in the composition is refolded.

12. The method according to claim 1, wherein said refolded NspA protein is derived from Neisseria meningitidis.

13. The method according to claim 1, wherein said refolded NspA protein is derived from Neisseria gonorrhoeae.

14. The medicament according to claim 10 wherein said medicament comprises at least one other Neisserial antigen.

15. The medicament according to claim 14 wherein said at least one other Neisserial antigen is selected from the group consisting of:
a. at least one Neisserial adhesin selected from the group consisting of FhaB, Hsf,NadA, Pi1C, Hap, MafA, MafB, Omp26, NMB0315, NMB0995 and NMB1119;
b. at least one Neisserial autotransporter selected from the group consisting of Hsf, Hap, IgA protease, AspA and NadA;
c. at least one Neisserial toxin selected from the group consisting of FrpA, FrpC, FrpA/C, VapD, NM-ADPRT, and either or both of LPS immunotype L2 and LPS immunotype L3;
d. at least one Neisserial Fe acquisition protein selected from the group consisting of TbpA high, TbpA low, TbpB high, TbpB low, LbpA, LbpB, P2086, HpuA, HpuB, Lipo28, Sibp, FbpA, BfrA, BfrB, Bcp, NMB0964 and NMB0293; and
e. at least one Neisserial membrane associated protein, preferably outer membrane protein, selected from the group consisting of P1dA, TspA, FhaC, TbpA(high), TbpA(low), LbpA, HpuB, TdfH, PorB, HimD, H isD, GNA1870, OstA, H1pA, M1tA, NMB 1124, NMB 1162, NMB 1220, NMB 1313, NMB 1953, HtrA, TspB, Pi1Q and OMP85.

16. The medicament of claim 10 further comprising one or more bacterial capsular polysaccharides or oligosaccharides.

17. The medicament of claim 16 wherein said one or more capsular polysaccharides or oligosaccharides are derived from bacteria selected from the group consisting of Neisseria meningitidis serogroup A, C, Y, and/or W-135, Haemophilus influenzae b, Streptococcus pneumoniae, Group A Streptococci, Group B Streptococci, Staphylococcus aureus and Staphylococcus epidermidis, and are preferably conjugated to a source of T-helper epitopes.


SEQUENCE LISTING (8 sequences)

SEQ ID NO: 1 - protein, 8 aa, Artificial Sequence
  VARIANT (2)...(2): Xaa is any amino acid
LXGGBGDX

SEQ ID NO: 2 - protein, 10 aa, Artificial Sequence
  Antiendotoxin peptide SAEP 2
KTKCKFLKKC

SEQ ID NO: 3 - DNA, 32 bp, Artificial Sequence
  PCR primer for amplifying NspA
gctacatatggaaggcgcatccggcttttacg

SEQ ID NO: 4 - DNA, 33 bp, Artificial Sequence
  PCR primer for amplifying NspA
gctaggatcctcagaatttgacgcgcacaccgg

SEQ ID NO: 5 - DNA, 468 bp, Neisseria meningitidis
gaaggcgcatccggcttttacgtccaagccgatgccgcacacgcaaaagcctcaagctctttaggttctgccaaaggcttcagcccgcgcatctccgcaggctaccgcatcaacgacctccgcttcgccgtcgattacacgcgctacaaaaactataaagccccatccaccgatttcaaactttacagcatcggcgcgtccgccatttacgacttcgacacccaatcgcccgtcaaaccgtatctcggcgcgcgcttgagcctcaaccgcgcctccgtcgacttgggcggcagcgacagcttcagccaaacctccatcggcctcggcgtattgacgggcgtaagctatgccgttaccccgaatgtcgatttggatgccggctaccgctacaactacatcggcaaagtcaacactgtcaaaaacgtccgttccggcgaactgtccgccggtgtgcgcgtcaaattctga

SEQ ID NO: 6 - protein, 155 aa, Neisseria meningitidis
EGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 7 - DNA, 525 bp, Neisseria meningitidis
atgaaaaaagcacttgccacactgattgccctcgctctcccggccgccgcactggcggaaggcgcatccggcttttacgtccaagccgatgccgcacacgcaaaagcctcaagctctttaggttctgccaaaggcttcagcccgcgcatctccgcaggctaccgcatcaacgacctccgcttcgccgtcgattacacgcgctacaaaaactataaagccccatccaccgatttcaaactttacagcatcggcgcgtccgccatttacgacttcgacacccaatcgcccgtcaaaccgtatctcggcgcgcgcttgagcctcaaccgcgcctccgtcgacttgggcggcagcgacagcttcagccaaacctccatcggcctcggcgtattgacgggcgtaagctatgccgttaccccgaatgtcgatttggatgccggctaccgctacaactacatcggcaaagtcaacactgtcaaaaacgtccgttccggcgaactgtccgtcggcgtgcgcgtcaaattctga

SEQ ID NO: 8 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSVGVRVKF